SEQUENCE LISTING (89 sequences)

SEQ ID NO: 1 - protein, 27 aa, synthetic construct
QVKLEESGGGLVQPGGSLRLSCAASGF

SEQ ID NO: 2 - protein, 27 aa, synthetic construct
QVQLVESGGGLVQPGGSLRLSCTASGF

SEQ ID NO: 3 - protein, 27 aa, synthetic construct
HVQLVESGGGLVQPGGSLTLSCAASGF

SEQ ID NO: 4 - protein, 27 aa, synthetic construct
QVQLVESGGGLVQPGGSLKLSCASSES

SEQ ID NO: 5 - protein, 27 aa, synthetic construct
QVDVQLVESGGGLVQPGGSLRLSCAAS

SEQ ID NO: 6 - protein, 27 aa, synthetic construct
QVKLEESGGGLVQPGGSLRLSCTASGF

SEQ ID NO: 7 - protein, 27 aa, synthetic construct
EVQLVESGGGLVQPGGSLTLSCAASGF

SEQ ID NO: 8 - protein, 27 aa, synthetic construct
QVQLVESGGGLVQPGGSLTLSCAASGF

SEQ ID NO: 9 - protein, 27 aa, synthetic construct
QVKLEESGGGLAQPGGSLRLSCTASGF

SEQ ID NO: 10 - protein, 27 aa, synthetic construct
DVQLVDSGGGLVQPGGSLRLSCSASGF

SEQ ID NO: 11 - protein, 27 aa, synthetic construct
QVKLEESGGGLAQPGGSLRLSCAASGF

SEQ ID NO: 12 - protein, 27 aa, synthetic construct
QVQLVESGGGLVQPGGSLRLSCAASGF

SEQ ID NO: 13 - protein, 27 aa, synthetic construct
AVQLVESGGGLVQPGGSLRLSCSASGF

SEQ ID NO: 14 - protein, 25 aa, synthetic construct
QVKLEESGGGVVQDGGSLRLSCAAI

SEQ ID NO: 15 - protein, 27 aa, synthetic construct
QVQLVESGGGLVQPGGSLRLSCSASGF

SEQ ID NO: 16 - protein, 25 aa, synthetic construct
DVQLVESGGGLVQPGGSLRLSCAAS

SEQ ID NO: 17 - protein, 8 aa, synthetic construct
SFSFKNYA

SEQ ID NO: 18 - protein, 8 aa, synthetic construct
SFSFENYA

SEQ ID NO: 19 - protein, 8 aa, synthetic construct
IFAFKNYA

SEQ ID NO: 20 - protein, 8 aa, synthetic construct
GFSFSFKN

SEQ ID NO: 21 - protein, 8 aa, synthetic construct
SFAFKDYA

SEQ ID NO: 22 - protein, 8 aa, synthetic construct
SFAFKNYA

SEQ ID NO: 23 - protein, 8 aa, synthetic construct
SSAFKNYA

SEQ ID NO: 24 - protein, 8 aa, synthetic construct
EQFFTTNA

SEQ ID NO: 25 - protein, 8 aa, synthetic construct
GFSFSFEN

SEQ ID NO: 26 - protein, 17 aa, synthetic construct
MSWVRQAPGKGLEWVST

SEQ ID NO: 27 - protein, 17 aa, synthetic construct
YAMTWVRQAPGKGLEWV

SEQ ID NO: 28 - protein, 17 aa, synthetic construct
MAWFRQAPGKERELVAA

SEQ ID NO: 29 - protein, 17 aa, synthetic construct
YAMSWVRQAPGKGLEWV

SEQ ID NO: 30 - protein, 8 aa, synthetic construct
MSGGGDET

SEQ ID NO: 31 - protein, 8 aa, synthetic construct
MSGGGGDT

SEQ ID NO: 32 - protein, 8 aa, synthetic construct
MSGGGGDI

SEQ ID NO: 33 - protein, 10 aa, synthetic construct
STMSGGGDET

SEQ ID NO: 34 - protein, 8 aa, synthetic construct
MSGSGGDT

SEQ ID NO: 35 - protein, 8 aa, synthetic construct
TDWSGQST

SEQ ID NO: 36 - protein, 10 aa, synthetic construct
STMSGGGGDT

SEQ ID NO: 37 - protein, 38 aa, synthetic construct
KYADSVKGRFTISRDNTKNTLYLQMNSLKPEDTAVYYC

SEQ ID NO: 38 - protein, 38 aa, synthetic construct
KYADSVKGRFTISRDNAKNTLYLQMNSLKPEDTAVYYC

SEQ ID NO: 39 - protein, 38 aa, synthetic construct
KYADSVKGRFTISRDNAKNTLYLQMNNLKPEDSAVYYC

SEQ ID NO: 40 - protein, 38 aa, synthetic construct
KYADSVKGRFTISRDNAKNTVYLQMNSLKPEDTAVYYC

SEQ ID NO: 41 - protein, 38 aa, synthetic construct
KYADSVKGRFTISRDNNKNTVYLQMNSLKPEDTAVYYC

SEQ ID NO: 42 - protein, 38 aa, synthetic construct
KYADSVKGRFTISRDNAKNAVYLQMNSLKPEDTAVYYC

SEQ ID NO: 43 - protein, 38 aa, synthetic construct
SYADSVKGRFTISRDTAKNVMYLQMNILHTEDTAVYYC

SEQ ID NO: 44 - protein, 38 aa, synthetic construct
KYADSVKGRFTISRDNAKNMLYLQMNSLKPEDTAVYYC

SEQ ID NO: 45 - protein, 14 aa, synthetic construct
AKGWITTDRFTNTP

SEQ ID NO: 46 - protein, 14 aa, synthetic construct
AKGWITTDRFTDTP

SEQ ID NO: 47 - protein, 14 aa, synthetic construct
AKGWITTNRFTNTP

SEQ ID NO: 48 - protein, 14 aa, synthetic construct
AKGWITTDQFASAP

SEQ ID NO: 49 - protein, 14 aa, synthetic construct
AKGWITTNQFADAP

SEQ ID NO: 50 - protein, 14 aa, synthetic construct
AKGWITTNRLTNTP

SEQ ID NO: 51 - protein, 14 aa, synthetic construct
AKGWITTNRFTNIP

SEQ ID NO: 52 - protein, 14 aa, synthetic construct
AEGWITTDQFASAP

SEQ ID NO: 53 - protein, 14 aa, synthetic construct
AKGWITNNQFASAP

SEQ ID NO: 54 - protein, 14 aa, synthetic construct
AKGWITTNQFTNTP

SEQ ID NO: 55 - protein, 14 aa, synthetic construct
AKGWIATDQFTNTP

SEQ ID NO: 56 - protein, 14 aa, synthetic construct
AKGWITTDQFTNTP

SEQ ID NO: 57 - protein, 14 aa, synthetic construct
AEGWITNNQFASAP

SEQ ID NO: 58 - protein, 14 aa, synthetic construct
AKGWITTDQFATAP

SEQ ID NO: 59 - protein, 14 aa, synthetic construct
AKGWITANRFTNTP

SEQ ID NO: 60 - protein, 14 aa, synthetic construct
AKGWITTDRFTDAP

SEQ ID NO: 61 - protein, 18 aa, synthetic construct
ARAPGRVLLTSDLESYTI

SEQ ID NO: 62 - protein, 14 aa, synthetic construct
AKGWITNNQFAFAP

SEQ ID NO: 63 - protein, 14 aa, synthetic construct
AKGWITTDRFTNTL

SEQ ID NO: 64 - protein, 11 aa, synthetic construct
RGQGTQVTVSS

SEQ ID NO: 65 - protein, 11 aa, synthetic construct
RGHGTQVTVSS

SEQ ID NO: 66 - protein, 11 aa, synthetic construct
WGQGTQVTVSS

SEQ ID NO: 67 - protein, 123 aa, synthetic construct
QVKLEESGGGLVQPGGSLRLSCAASGFSFSFKNYAMSWVRQAPGKGLEWVSTMSGGGDETKYADSVKGRFTISRDNTKNTLYLQMNSLKPEDTAVYYCAKGWITTDRFTNTPRGQGTQVTVSS

SEQ ID NO: 68 - protein, 123 aa, synthetic construct
QVQLVESGGGLVQPGGSLRLSCTASGFSFSFKNYAMSWVRQAPGKGLEWVSTMSGGGGDTKYADSVKGRFTISRDNAKNTLYLQMNSLKPEDTAVYYCAKGWITTDRFTDTPRGQGTQVTVSS

SEQ ID NO: 69 - protein, 123 aa, synthetic construct
HVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRFTNTPRGQGTQVTVSS

SEQ ID NO: 70 - protein, 123 aa, synthetic construct
QVQLVESGGGLVQPGGSLKLSCASSESIFAFKNYAMSWVRQAPGKGLEWVSTMSGGGGDTKYADSVKGRFTISRDNAKNTVYLQMNSLKPEDTAVYYCAKGWITTDQFASAPRGHGTQVTVSS

SEQ ID NO: 71 - protein, 125 aa, synthetic construct
QVDVQLVESGGGLVQPGGSLRLSCAASGFSFSFKNYAMTWVRQAPGKGLEWVSTMSGGGDETKYADSVKGRFTISRDNTKNTLYLQMNSLKPEDTAVYYCAKGWITTDRFTNTPRGQGTQVTVSS

SEQ ID NO: 72 - protein, 123 aa, synthetic construct
QVKLEESGGGLVQPGGSLRLSCTASGFSFAFKDYAMSWVRQAPGKGLEWVSTMSGGGGDTKYADSVKGRFTISRDNNKNTVYLQMNSLKPEDTAVYYCAKGWITTNQFADAPRGQGTQVTVSS

SEQ ID NO: 73 - protein, 123 aa, synthetic construct
EVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRLTNTPRGQGTQVTVSS

SEQ ID NO: 74 - protein, 123 aa, synthetic construct
QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRFTNIPRGQGTQVTVSS

SEQ ID NO: 75 - protein, 123 aa, synthetic construct
QVKLEESGGGLAQPGGSLRLSCTASGFSFAFKNYAMSWVRQAPGKGLEWVSTMSGGGGDTKYADSVKGRFTISRDNAKNTVYLQMNSLKPEDTAVYYCAKGWITTDQFASAPRGHGTQVTVSS

SEQ ID NO: 76 - protein, 123 aa, synthetic construct
QVQLVESGGGLVQPGGSLRLSCTASGFSFAFKNYAMSWVRQAPGKGLEWVSTMSGGGGDTKYADSVKGRFTISRDNAKNTVYLQMNSLKPEDTAVYYCAEGWITTDQFASAPRGQGTQVTVSS

SEQ ID NO: 77 - protein, 123 aa, synthetic construct
DVQLVDSGGGLVQPGGSLRLSCSASGFSSAFKNYAMSWVRQAPGKGLEWVSTMSGGGGDTKYADSVKGRFTISRDNNKNTVYLQMNSLKPEDTAVYYCAKGWITNNQFASAPRGQGTQVTVSS

SEQ ID NO: 78 - protein, 123 aa, synthetic construct
QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNQFTNTPRGQGTQVTVSS

SEQ ID NO: 79 - protein, 123 aa, synthetic construct
QVKLEESGGGLAQPGGSLRLSCAASGFSFSFKNYAMSWVRQAPGKGLEWVSTMSGGGGDIKYADSVKGRFTISRDNAKNTLYLQMNSLKPEDTAVYYCAKGWIATDQFTNTPRGQGTQVTVSS

SEQ ID NO: 80 - protein, 123 aa, synthetic construct
QVQLVESGGGLVQPGGSLRLSCAASGFSFSFKNYAMSWVRQAPGKGLEWVSTMSGGGGDIKYADSVKGRFTISRDNAKNTLYLQMNSLKPEDTAVYYCAKGWITTDQFTNTPRGQGTQVTVSS

SEQ ID NO: 81 - protein, 123 aa, synthetic construct
AVQLVESGGGLVQPGGSLRLSCSASGFSFAFKNYAMSWVRQAPGKGLEWVSTMSGGGGDTKYADSVKGRFTISRDNNKNTVYLQMNSLKPEDTAVYYCAEGWITNNQFASAPRGQGTQVTVSS

SEQ ID NO: 82 - protein, 123 aa, synthetic construct
QVQLVESGGGLVQPGGSLRLSCTASGFSFAFKNYAMSWVRQAPGKGLEWVSTMSGGGGDTKYADSVKGRFTISRDNAKNAVYLQMNSLKPEDTAVYYCAKGWITTDQFATAPRGQGTQVTVSS

SEQ ID NO: 83 - protein, 123 aa, synthetic construct
QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITANRFTNTPRGQGTQVTVSS

SEQ ID NO: 84 - protein, 123 aa, synthetic construct
QVQLVESGGGLVQPGGSLRLSCAASGFSFSFKNYAMSWVRQAPGKGLEWVSTMSGSGGDTKYADSVKGRFTISRDNAKNTLYLQMNSLKPEDTAVYYCAKGWITTDRFTDAPRGQGTQVTVSS

SEQ ID NO: 85 - protein, 125 aa, synthetic construct
QVKLEESGGGVVQDGGSLRLSCAAIEQFFTTNAMAWFRQAPGKERELVAATDWSGQSTSYADSVKGRFTISRDTAKNVMYLQMNILHTEDTAVYYCARAPGRVLLTSDLESYTIWGQGTQVTVSS

SEQ ID NO: 86 - protein, 123 aa, synthetic construct
QVQLVESGGGLVQPGGSLRLSCSASGFSFAFKNYAMSWVRQAPGKGLEWVSTMSGGGGDTKYADSVKGRFTISRDNNKNTVYLQMNSLKPEDTAVYYCAKGWITNNQFAFAPRGQGTQVTVSS

SEQ ID NO: 87 - protein, 123 aa, synthetic construct
DVQLVESGGGLVQPGGSLRLSCAASGFSFSFENYAMSWVRQAPGKGLEWVSTMSGGGGDTKYADSVKGRFTISRDNAKNMLYLQMNSLKPEDTAVYYCAKGWITTDRFTNTLRGQGTQVTVSS

SEQ ID NO: 88 - protein, 394 aa, synthetic construct
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 89 - protein, 393 aa, synthetic construct
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

(19) United States
(12) Patent Application Publication
(10) Pub. No.: US 2023/0279077 A1
(43) Pub. Date: Sep. 7, 2023

(54) ANTI-GLYCOPROTEIN D ANTIBODIES, METHODS OF PREPARATION, AND USES THEREOF

(71) Applicant: Virogin Biotech Canada Ltd., Richmond (CA)

(72) Inventors: Xiaohu LIU, Coquitlam (CA); Yanal M MURAD, Vancouver (CA)

(21) Appl. No.: 18/159,473

(22) Filed: Jan. 25, 2023

Related U.S. Application Data
(60) Provisional application No. 63/303,067, filed on Jan. 26, 2022.

(51) Int. Cl. C07K 16/08; A61P 31/22; G01N 33/569

(52) U.S. Cl. CPC C07K 16/087; A61P 31/22; G01N 33/56994; C07K 2317/31; C07K 2317/76; C07K 2317/92; C07K 2317/569

(57) ABSTRACT

The present disclosure provides antibodies that bind to a gD protein. The present disclosure also discloses a composition comprising an anti-gD protein antibody, a pharmaceutical composition comprising an anti-gD protein antibody and a pharmaceutically acceptable carrier. Also disclosed are a polynucleotide sequence encoding an anti-gD protein, a vector comprising such a polynucleotide, and a cell capable of expressing an anti-gD protein. The present disclosure also provides a method for producing an anti-gD protein antibody, a method for treating HSV-1 and/or HSV-2 infection, and a method for detecting HSV-1 and/or HSV-2.

### CROSS-REFERENCE TO RELATED APPLICATION

This application claims priority to U.S. Provisional Application No. 63/303,067, filed on Jan. 26, 2022, the contents of which are hereby incorporated by reference.

### SEQUENCE LISTING

This application contains a Sequence Listing as an XML file entitled “157450002-00000 US.xml” having a size of 81,899 bytes and created on Jan. 24, 2023. The information contained in the Sequence Listing is incorporated by reference herein.

### FIELD OF DISCLOSURE

The present disclosure relates to antibodies targeting a glycoprotein D (gD) protein. The present disclosure also discloses compositions, pharmaceutical compositions, polynucleotides, vectors, and cells related to anti-gD protein antibodies. Also provided are methods to produce and use anti-gD protein antibodies.

### BACKGROUND

Millions of people worldwide are exposed to herpes simplex virus (HSV). HSV-1 infections are primarily associated with mild to severe symptoms including blisters and inflammation of oral and ocular cells but in some cases, and the infection can progress to more serious illnesses including blindness, hearing impairment, and fatal encephalitis. HSV-2 infection is widespread throughout the world and is almost exclusively sexually transmitted, causing genital herpes.

HSV has been explored for cancer treatment because it has several advantages as a template for viral oncolysis vectors (Carson et al., 2010). The relatively large genome of HSV allows it to accept multiple transgenes as large as 30 kb. As the HSV genome does not integrate into the host genome, it is generally nonmutagenic to its host. In addition, the existence of multiple antiviral medications with well-established safety and efficacy in the treatment of HSV infections provides a reassuring option for certain scenarios where oncolytic treatments were to lead to an unexpected pathogenic infection.

Antibodies are used extensively as therapeutics agents as well as tools for developing new therapies. Antibodies against HSV are needed for HSV infection treatment as well as for developing tools to study and develop new therapies utilizing HSV-based oncolytic virus. There is a need for both neutralizing and non-neutralizing HSV antibodies.

### SUMMARY

The present disclosure provides antibodies that bind to a gD protein. The multiple antibodies provided herein can bind different regions of the gD protein, and thus they may have different effects on the biology and function of this glycoprotein in the infectivity of HSV (e.g., HSV-1 or HSV-2). These antibodies include both neutralizing and non-neutralizing antibodies, which generate a toolbox to choose from for different applications.

In an aspect, the present disclosure provides an antibody comprising a heavy chain variable region, wherein the heavy chain variable region comprises three complementarity determining regions (CDRs), designated as CDR1, CDR2, and CDR3, wherein the CDR1 is selected from SEQ ID NOs: 17-25, the CDR2 is selected from SEQ ID NOs:30-36, and the CDR3 is selected from SEQ ID NOs:45-63, and wherein the antibody binds a gD protein.

In some embodiments, the antibody described herein comprises the CDR1, CDR2, and CDR3 that comprise:
(a) SEQ ID NO: 17, SEQ ID NO:30, and SEQ ID NO:45, respectively;
(b) SEQ ID NO: 17, SEQ ID NO:31, and SEQ ID NO:46, respectively;
(c) SEQ ID NO: 18, SEQ ID NO:32, and SEQ ID NO:47, respectively;
(d) SEQ ID NO: 19, SEQ ID NO:31, and SEQ ID NO:48, respectively;
(e) SEQ ID NO:20, SEQ ID NO:33, and SEQ ID NO:45, respectively;
(f) SEQ ID NO:21, SEQ ID NO:31, and SEQ ID NO:49, respectively;
(g) SEQ ID NO: 18, SEQ ID NO:32, and SEQ ID NO:50, respectively;
(h) SEQ ID NO:18, SEQ ID NO:32, and SEQ ID NO:51, respectively;
(i) SEQ ID NO:22, SEQ ID NO:31, and SEQ ID NO:48, respectively;
(j) SEQ ID NO:22, SEQ ID NO:31, and SEQ ID NO:52, respectively;
(k) SEQ ID NO:23, SEQ ID NO:31, and SEQ ID NO:53, respectively;
(l) SEQ ID NO:18, SEQ ID NO:32, and SEQ ID NO:54, respectively;
(m) SEQ ID NO:17, SEQ ID NO:32, and SEQ ID NO:55, respectively;
(n) SEQ ID NO:17, SEQ ID NO:32, and SEQ ID NO:56, respectively;
(o) SEQ ID NO:22, SEQ ID NO:31, and SEQ ID NO:57, respectively;
(p) SEQ ID NO:22, SEQ ID NO:31, and SEQ ID NO:58, respectively;
(q) SEQ ID NO:18, SEQ ID NO:32, and SEQ ID NO:59, respectively;
(r) SEQ ID NO:17, SEQ ID NO:34, and SEQ ID NO:60, respectively;
(s) SEQ ID NO:24, SEQ ID NO:35, and SEQ ID NO:61, respectively;
(t) SEQ ID NO:22, SEQ ID NO:31, and SEQ ID NO:62, respectively; or
(u) SEQ ID NO:25, SEQ ID NO:36, and SEQ ID NO:63, respectively.

In some embodiments, the antibody described herein comprises a framework region 1 (FR1) selected from SEQ ID NOs:1-16, a framework region 2 (FR2) selected from SEQ ID NOs:26-29, a framework region 3 (FR3) selected from SEQ ID NOs:37-44, and a framework region 4 (FR4) selected from SEQ ID NOs:64-66.

In some embodiments, the antibody described herein comprises the FR1, FR2, FR3, and FR4 that comprise
(a) SEQ ID NO:1, SEQ ID NO:26, SEQ ID NO:37, and SEQ ID NO:64, respectively;
(b) SEQ ID NO:2, SEQ ID NO:26, SEQ ID NO:38, and SEQ ID NO:64, respectively;
(c) SEQ ID NO:3, SEQ ID NO:26, SEQ ID NO:39, and SEQ ID NO:64, respectively;
(d) SEQ ID NO:4, SEQ ID NO:26, SEQ ID NO:40, and SEQ ID NO:65, respectively;
(e) SEQ ID NO:5, SEQ ID NO:27, SEQ ID NO:37, and SEQ ID NO:64, respectively;
(f) SEQ ID NO:6, SEQ ID NO:26, SEQ ID NO:41, and SEQ ID NO:64, respectively;
(g) SEQ ID NO:7, SEQ ID NO:26, SEQ ID NO:39, and SEQ ID NO:64, respectively;
(h) SEQ ID NO:8, SEQ ID NO:26, SEQ ID NO:39, and SEQ ID NO:64, respectively;
(i) SEQ ID NO:9, SEQ ID NO:26, SEQ ID NO:40, and SEQ ID NO:65, respectively;
(j) SEQ ID NO:2, SEQ ID NO:26, SEQ ID NO:40, and SEQ ID NO:64, respectively;
(k) SEQ ID NO:10, SEQ ID NO:26, SEQ ID NO:41, and SEQ ID NO:64, respectively;
(l) SEQ ID NO:8, SEQ ID NO:26, SEQ ID NO:39, and SEQ ID NO:64, respectively;
(m)SEQ ID NO:11, SEQ ID NO:26, SEQ ID NO:38, and SEQ ID NO:64, respectively;
(n) SEQ ID NO:12, SEQ ID NO:26, SEQ ID NO:38, and SEQ ID NO:64, respectively;
(o) SEQ ID NO:13, SEQ ID NO:26, SEQ ID NO:41, and SEQ ID NO:64, respectively;
(p) SEQ ID NO:2, SEQ ID NO:26, SEQ ID NO:42, and SEQ ID NO:64, respectively;
(q) SEQ ID NO:8, SEQ ID NO:26, SEQ ID NO:39, and SEQ ID NO:64, respectively;
(r) SEQ ID NO:12, SEQ ID NO:26, SEQ ID NO:38, and SEQ ID NO:64, respectively;
(s) SEQ ID NO:14, SEQ ID NO:28, SEQ ID NO:43, and SEQ ID NO:66, respectively;
(t) SEQ ID NO:15, SEQ ID NO:26, SEQ ID NO:41, and SEQ ID NO:64, respectively; or
(u) SEQ ID NO:16, SEQ ID NO:29, SEQ ID NO:44, and SEQ ID NO:64, respectively.

In some embodiments, the antibody described herein comprises a heavy chain variable domain, wherein the heavy chain variable domain comprises an amino acid sequence as set forth in any one of SEQ ID NOs:67-87. In some embodiments, the antibody described herein comprises an amino acid sequence with at least 80%, 85%, 90%, 95%, 96%, 97%, 98%, 99%, or 100% identity to any one of SEQ ID NOs:67-87.

In some embodiments, the gD protein that the antibody described herein specifically binds to is derived from a virus. In some embodiments, the virus is an HSV. In some embodiments, the HSV is an HSV-1 or an HSV-2.

In some embodiments, the gD protein that the antibody described herein specifically binds to is a recombinant gD protein.

In some embodiments, the antibody described herein binds the gD protein with a K_{D} ranging from about 1 pM to about 1 µM. In some embodiments, the K_{D} ranges from about 50 pM to about 4 nM. In some embodiments, the K_{D} ranges from about 0.05 nM to 0.2 nM. In some embodiments, the K_{D} is about 4 nM or lower.

In some embodiments, the antibody described herein is a single domain antibody (sdAb). In some embodiments, the antibody described herein is a single heavy domain antibody.

In some embodiments, the antibody described herein is a neutralizing antibody. In some embodiments, the antibody described herein is capable of neutralizing HSV-1 and/or HSV-2. In some embodiments, the neutralizing antibody comprises an amino acid sequence as set forth in SEQ ID NO:85. In some embodiments, the neutralizing antibody comprises an amino acid sequence with at least 90%, 95%, 96%, 97%, 98%, 99%, or 100% identity to SEQ ID NO:85.

In some embodiments, the antibody described herein is a multispecific antibody. In some embodiments, the multispecific antibody comprises an amino acid sequence set forth in SEQ ID NO:85 and one or more amino acid sequences selected from SEQ ID NOs:67-84 and 86-87.

In some embodiments the antibody described herein is a non-neutralizing antibody.

In another aspect, the present disclosure provides a composition comprising the antibody described herein. In some embodiments, the present disclosure provides a pharmaceutical composition comprising the antibody described herein and a pharmaceutically acceptable carrier.

In another aspect, the present disclosure provides a polynucleotide that encodes the antibody described herein.

In another aspect, the present disclosure provides a vector comprising the polynucleotide as described herein.

In another aspect, the present disclosure provides a cell capable of expressing the antibody described herein. In another aspect, the present disclosure provides a cell comprising the polynucleotide described herein or the vector described herein.

In another aspect, the present disclosure provides a method of producing an antibody, comprising culturing the cell described herein and recovering the antibody from the cell.

In another aspect, the present disclosure provides a method for treating infection of HSV-1 and/or HSV-2 in a subject, comprising administering to the subject an effective amount of the antibody as described herein or the composition as described herein.

In another aspect, the present disclosure provides a method for detecting HSV-1 and/or HSV-2 in a sample, comprising contacting the sample with the antibody described herein or the composition described herein, and detecting the presence of the antibody.

### BRIEF DESCRIPTION OF THE DRAWINGS

FIG. 1 provides results of serum response monitoring. Raw ELISA data comparing pre-immune (Pre) and immune (d42) serum for binding to inactivated HSV-1 (“E”) and glycoprotein D (“G”). Detection of total polyclonal response (conventional and heavy-chain IgG) with anti-llama Fc IgG (FIG. 1A), or detection of heavy-chain IgG with a hinge-specific mAb 1C10 (FIG. 1B).

FIG. 2 provides results of Phage ELISA on round-3 eluted phage. FIG. 2A shows a 96-well plate layout, and FIG. 2B shows A450 nm values for specific round-3 clones. PBS: negative control; helper phage: positive control. All clones were DNA sequenced, the sequences were analyzed, and certain VHHs were selected for soluble VHH expression and characterization.

FIG. 3A provides SEC profiles of anti-glycoprotein D VHHs. FIG. 3B provides calibration results of a S200 INCREASE column.

FIG. 4 provides results of immobilization of anti-human IgG.

FIG. 5 provides a SPR sensorgram demonstrating high-affinity binding of the G44 VHH to a fusion glycoprotein D (referred to as “G-Fc”). Single-cycle kinetics were used to determined kinetics and affinities by injecting G44 at concentrations ranging from 0.125 nM to 2 nM over the glycoprotein D-Fc surface.

FIG. 6 provides SRP sensorgram results showing binding of sdAbs to captured G-Fc.

FIG. 7 provides a summary of SPR-based epitope binning results. VHHs G19, G41, G44, and G77 bind overlapping or competing epitopes on glycoprotein D. VHH G75 binds a non-overlapping or distinct epitope from these other set of VHHs.

FIG. 8 provides SRP sensorgram results for epitope binning.

### DETAILED DESCRIPTION

The disclosures and embodiments set forth herein are to be construed as exemplary only and not as limiting the scope of the invention. Although specific terms are employed herein, unless otherwise noted, they are used in a generic and descriptive sense only and not for purposes of limitation.

All publications, patents, and patent applications referred to herein are incorporated by reference in their entirety to the same extent as if each individual publication, patent, or patent application was specifically and individually indicated to be incorporated by reference in its entirety.

### Definitions

In the present disclosure, unless otherwise specified, the scientific and technical terms used herein have the meanings generally understood by a person skilled in the art. Although any methods and materials similar or equivalent to those described herein find use in the practice of the present disclosure, the preferred methods and materials are described herein. Accordingly, the terms defined herein are more fully described by reference to the Specification as a whole.

Unless defined otherwise, all technical and scientific terms, acronyms, and abbreviations used herein have the same meaning as commonly understood by one of ordinary skill in the art to which the disclosure pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present disclosure also contemplates that in some embodiments of the disclosure, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

Unless otherwise indicated, nucleic acids are written left to right in the 5′ to 3′ orientation; and amino acid sequences are written left to right in amino to carboxy orientation, respectively.

It is to be understood that this disclosure is not limited to the particular methodology, protocols, and reagents described, as these may vary, depending upon the context in which they are used by those skilled in the art.

As used herein, the term “about” will be understood by persons of ordinary skill in the art and will vary to some extent depending on the context in which it is used. In some embodiments, the term “about” when referring to a measurable value such as an amount, a temporal duration, and the like, is meant to encompass art-accepted variations based on standard errors in making such measurements. In some embodiments, the term “about” when referring to such values, is meant to encompass variations of ±20% or ±10%, more preferably ±5%, even more preferably ±1%, and still more preferably ±0.1% from the specified value, as such variations are appropriate to perform the disclosed methods.

As used herein, the terms “percent identity” and “% identity,” as applied to nucleic acid or polynucleotide sequences, refer to the percentage of residue matches between at least two nucleic acid or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between nucleic acid or polynucleotide sequences may be determined using a suite of commonly used and freely available sequence comparison algorithms provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/.

Nucleic acid or polynucleotide sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. Specifically, degenerate codon substitutions may be achieved by generating sequences in which the third position of one or more selected (or all) codons is substituted with mixed-base and/or deoxyinosine residues (Batzer et al., 1991) Nucleic Acid Res 19:5081; Ohtsuka et al. (1985) J Biol Chem 260:2605-2608; Cassol et al. (1992); Rossolini et al. (1994) Mol Cell Probes 8:91-98). The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The term nucleic acid is used interchangeably with polynucleotide, and (in appropriate contexts) gene, cDNA, and mRNA encoded by a gene.

As used herein, “percent (%) amino acid sequence identity” with respect to a peptide, polypeptide or protein sequence is defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Percent amino acid sequence identity in the current disclosure is measured using BLAST software. Those skilled in the art can determine appropriate parameters for measuring alignment, including any algorithms needed to achieve maximal alignment over the full length of the sequences being compared.

An amino acid substitution refers to the replacement of one amino acid in a polypeptide with another amino acid. Exemplary substitutions are shown in Table 1. Amino acid substitutions may be introduced into a protein of interest and the products screened for a desired activity, for example, retained/improved biological activity.

TABLE 1

Exemplary Amino Acid Substitutions

| | |
|---|---|
| Original Residue | Exemplary Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. The term, “corresponding to” with reference to nucleotide or amino acid positions of a sequence, such as set forth in the Sequence Listing, refers to nucleotides or amino acid positions identified upon alignment with a target sequence based on structural sequence alignment or using a standard alignment algorithm, such as the GAP algorithm. For example, corresponding residues of a similar sequence (e.g., a fragment or species variant) can be determined by alignment to a reference sequence by structural alignment methods. By aligning the sequences, one skilled in the art can identify corresponding residues, for example, using conserved and identical amino acid residues as guides.

As used herein, the term “wild-type” means naturally occurring.

As used herein, the term “range,” “ranges,” or “ranging” from a scope means that the value at issue is equal or higher than the minimum value of the scope provided, and is equal or lower than the maximum value of the scope provided.

As used herein, “antibody” refers to any immunoglobulin (Ig) molecules, including but not limited to, monoclonal antibody, human antibody, non-human antibody, llama antibody, humanized antibody, chimeric antibody, single domain antibody, antibody fragments, antigen binding fragment, bispecific antibody, multispecific antibody, multimeric antibody, single chain antibody, or any functional fragment, mutant, variant, or derivation thereof, that specifically binds to or interacts with at least one particular antigen (e.g., HSV-1 gD protein, HSV-2 gD protein).

In humans and most mammals, an antibody unit typically consists of four polypeptide chains: two identical heavy chains and two identical light chains connected by disulfide bonds. Light chains consist of one variable domain VL and one constant domain CL, while heavy chains contain one variable domain VH and three to four constant domains, e.g., CH1, CH2, CH3. Immunoglobulins can be assigned to five major classes, namely IgA, IgD, IgE, IgG, and IgM, depending on the heavy chain constant domain amino acid sequence. IgA and IgG are typically further sub-classified to IgA1, IgA2, IgG1, IgG2, IgG3, and IgG4. Antibody light chains of vertebrate species can be assigned to one of two distinct types, namely kappa (κ and lambda (λ), based on the amino acid sequences of their constant domains.

As used herein, “antigen binding fragment” or “antibody fragment” refers to a portion of an immunoglobulin molecule that retains the heavy chain and/or the light chain antigen binding site, such as a heavy chain complementarity determining region, a light chain complementarity determining region, a heavy chain variable region (VH), or a light chain variable region (VL). Antibody fragment includes, but not limited to, a Fab fragment (a monovalent fragment consisting of the VL or the VH), a F(ab)₂ fragment (a bivalent fragment comprising two Fab fragments linked by a disulfide bridge at the hinge region), a Fd fragment consisting of the VH and CH1 domains, a Fv fragment consisting of the VL and VH domains of a single arm of an antibody, a dAb fragment, which consists of a VH domain, or a variable domain (VHH) from, e.g., human or camelid origin. These antibody fragments can be obtained using well known techniques. VHH refers to an antigen binding fragment of heavy chain only antibodies.

An antibody variable domain consists of “framework” (FR) regions and “complementarity determining regions” (CDRs). For example, in some embodiments, a heavy chain variable domain of an antibody has a structure of FR1-CDR1-FR2-CDR2-FR3-CDR3-FR4.

CDR sequences are variable antibody sequences that respond with specificity, duration and strength to identify and bind to antigen epitopes. FR sequences are the remaining sequences of a variable region other than those sequences defined to be CDRs. CDR denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 Jan;27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun 8;309(3):657-70, (“Aho” numbering scheme); and Martin et al., “Modeling antibody hypervariable loops: a combined algorithm,” PNAS, 1989, 86(23):9268-9272, (“AbM” numbering scheme). In some embodiments, for example, the CDRs in the present application are determined based on the IMGT numbering scheme.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular’s AbM antibody modeling software. In some embodiments, CDRs can be defined in accordance with any of the Chothia numbering schemes, the Kabat numbering scheme, the IMGT numbering scheme, a combination of Kabat, IMGT, and Chothia, the AbM definition, and/or the contact definition.

As used herein, “affinity” refers to the strength of the sum total of noncovalent interactions between a single binding site of a molecule (e.g., a receptor) and its binding partner (e.g., a ligand). The affinity of a molecule for its partner can generally be represented by the equilibrium dissociation constant (K_{D}) (or its inverse equilibrium association constant, K_{A}). Affinity can be measured by common methods known in the art, including those described herein. See, for example, Pope M.E., Soste M.V., Eyford B.A., Anderson N.L., Pearson T.W., (2009) J. Immunol. Methods. 341(1-2):86-96 and methods described therein.

As used herein, the terms “specifically bind” or “bind” to a particular antigen refer to binding that is measurably different from a non-specific interaction. For example, in some embodiments, a binding molecule, such as a single domain antibody, specifically binds to a target molecule, such as an antigen, when the binding molecule reacts or associates more frequently, more rapidly, with greater duration, and/or with greater affinity with the particular target molecule than it does with alternative molecules. A binding molecule, such as a sdAb, “specifically binds” to a target molecule if it binds with greater affinity, avidity, more readily, and/or with greater duration than it binds to other molecules. It is understood that a binding molecule, such as a sdAb, that specifically binds to a first target may or may not specifically bind to a second target. As such, “specific binding” does not necessarily require (although it can include) exclusive binding. In some embodiments, specific binding can be determined, for example, by comparing binding of a particular antibody to binding of an antibody that does not bind to a particular antigen. Specific binding for a particular antigen can be shown, for example, when an antibody has a K_{D} for an antigen of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, at least about 10⁻¹² M, or greater, where K_{D} refers to a dissociation rate of the antibody/antigen interaction. In some embodiments, an antibody that specifically binds an antigen will have a K_{D} that is 20, 50, 100, 500, 1000, 5,000, 10,000 or more times greater than the K_{D} of an antibody that does not bind to the same antigen. In some embodiments, the binding between an antibody and a particular antigen can be shown by an EC50 value, determined using suitable methods known in the art, including, for example, flow cytometry assay.

As used herein, “epitope” refers to the part of an antigen that is recognized and bound by an antibody. An antigen may have more than one epitopes that are recognized by an antibody.

As used herein, a “composition” refers to any mixture of two or more products, substances, or compounds, including but not limited to, proteins, antibodies, polynucleotides, vectors, or cells. It may be a solution, a suspension, liquid, powder, a paste, aqueous, non-aqueous, or any combination thereof.

As used herein, a “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. Some non-limiting examples of the components that could be included in the composition are carriers, stabilizers, diluents, dispersing agents, suspending agents, thickening agents, and/or excipients. The pharmaceutical composition facilitates administration of the antibody or cell described herein to a subject. Multiple techniques of administration exist in the art including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of a therapeutic compound, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “effective amount” refers to an amount of a pharmaceutical composition which is sufficient to significantly and positively modify the symptoms and/or conditions to be treated (e.g., provide a positive clinical response). The effective amount of a pharmaceutical composition will vary with the particular condition being treated, the severity of the condition, the duration of treatment, the nature of concurrent therapy, the particular composition being employed, the particular pharmaceutically-acceptable excipient(s) and/or carrier(s) utilized, and like factors with the knowledge and expertise of the attending physician.

As used herein, a “disease” or “disorder” refers to a condition in which treatment is needed and/or desired.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

As used herein, the terms “individual” and “subject” are used interchangeably herein to refer to an animal. For example, in some embodiments, the animal is a mammal. In some embodiments, the animals are humans, rodents, simians, felines, canines, equines, bovines, porcines, ovines, caprines, mammalian laboratory animals, mammalian farm animals, mammalian sport animals, or mammalian pets. The animal can be male or female and can be at any suitable age, including infant, juvenile, adolescent, adult, and geriatric. In some examples, an “individual” or “subject” refers to an animal in need of treatment for a disease or disorder. In some embodiments, the animal to receive the treatment can be a “patient,” designating the fact that the animal has been identified as having a disorder of relevance to the treatment, or being at adequate risk of contracting the disorder. In particular embodiments, the animal is a human, such as a human patient.

### HSV and gD Protein

Herpes Simplex Virus (HSV, also known as HHV) are generally categorized into two types: HSV-1 and HSV-2. HSV belongs to the family Herpesviridae, which is classified into three subfamilies: alpha-herpesviruses, beta-herpesviruses, and gamma-herpesviruses subfamilies. HSV belongs to the alpha-herpesvirus subfamily, and generally has a short replicative cycle and is capable of infecting a broad host range. A mature HSV comprises the following: 1) a linear double stranded DNA of -152 kb encoding at least 74 genes, 2) encased in an icosapentahedral capsid composed of 162 capsomeres made of six different viral proteins, 3) surrounded by 20-23 different viral tegument proteins that have structural and regulatory roles, and 4) covered by an envelope that has at least 13 different glycoproteins, e.g., gB, gD, gH, gL, in distinct shapes and sizes, several of which are incorporated into mature virions. Of these viral glycoproteins, four glycoproteins (gB, gD and the heterodimer gH/gL) have been implicated in HSV fusion and entry.

The wild-type mature HSV-1 gD protein is a 369 amino acid residue long type I membrane glycoprotein that is approximately 8-10 nm long. HSV-1 gD is expressed with a 25 amino acid long signal peptide. The amino acid sequence of the wild-type HSV-1 gD protein is as set forth in SEQ ID NO:88 (UniProtKB - Q69091 (GD _HHV11)).

SEQ ID NO:88 (wild-type HSV-1 gD protein):MGGAAARL
GAVILFVVIVGLHGVRSKYALVDASLKMADPNRFRGKDLPVLDQLTDPPG
VRRVYHIQAGLPDPFQPPSLPITVYYAVLERACRSVLLNAPSEAPQIVRG
ASEDVRKQPYNLTIAWFRMGGNCAIPITVMEYTECSYNKSLGACPIRTQP
RWNYYDSFSAVSEDNLGFLMHAPAFETAGTYLRLVKINDWTEITQFILEH
RAKGSCKYALPLRIPPSACLSPQAYQQGVTVDSIGMLPRFIPENQRTVAV
YSLKIAGWHGPKAPYTSTLLPPELSETPNATQPELAPEDPEDSALLEDPV
GTVAPQIPPNWHIPSIQDAATPYHPPATPNNMGLIAGAVGGSLLAALVIC
GIVYWMRRHTQKAPKRIRLPHIREDDQPSSHQPLFY

The wild-type mature HSV-2 gD protein is a 363 amino acid residue long single-pass type I membrane glycoprotein. HSV-2 gD is expressed with a 30 amino acid long signal peptide. The amino acid sequence of the wild-type HSV-2 gD protein is as set forth in SEQ ID NO:89 (UniProtKB - P03172 (GD_HHV23)).

SEQ ID NO:89 (wild-type HSV-2 gD protein):MGRLTSGV
GTAALLVVAVGLRVVCAKYALADPSLKMADPNRFRGKNLPVLDRLTDPPG
VKRVYHIQPSLEDPFQPPSIPITVYYAVLERACRSVLLHAPSEAPQIVRG
ASDEARKHTYNLTIAWYRMGDNCAIPITVMEYTECPYNKSLGVCPIRTQP
RWSYYDSFSAVSEDNLGFLMHAPAFETAGTYLRLVKINDWTEITQFILEH
RARASCKYALPLRIPPAACLTSKAYQQGVTVDSIGMLPRFIPENQRTVAL
YSLKIAGWHGPKPPYTSTLLPPELSDTTNATQPELVPEDPEDSALLEDPA
GTVSSQIPPNWHIPSIQDVAPHHAPAAPSNPGLIIGALAGSTLAVLVIGG
IAFWVRRRAQMAPKRLRLPHIRDDDAPPSHQPLFY

HSV gD proteins are irregularly clustered on the viral membrane surface. HSV gD is typically organized into an ectodomain, a transmembrane domain and a short cytoplasmic tail. According to crystallographic studies, the gD ectodomain has an immunoglobulin-like core, edged by N and C terminal extensions on either ends. The N-terminus domain is termed as the receptor binding domain (RBD), and this part of the gD binds with specific host receptors. The C-terminus domain is termed as pro-fusion domain (PFD) which interact with gH/gL and gB.

### Antibodies

The present disclosure provides novel antibodies that specifically bind a gD protein.

In an aspect, the present disclosure provides an antibody comprising a heavy chain variable region, wherein the heavy chain variable region comprises three complementarity determining regions (CDRs), designated as CDR1, CDR2, and CDR3, wherein the CDR1 is selected from SEQ ID NOs: 17-25, the CDR2 is selected from SEQ ID NOs:30-36, and the CDR3 is selected from SEQ ID NOs:45-63, and wherein the antibody binds a gD protein.

As used herein, “gD protein” or “glycoprotein D” includes wild-type gD proteins in all isoforms, functional variants of a wild-type gD protein, precursors of a gD protein, recombinant gD proteins, synthesized gD proteins, purified gD proteins, isolated gD proteins, gD proteins that are fused with another protein, gD proteins that are conjugated with another entity, e.g., a label, a truncated form of any gD protein, or a functional fragment of any gD protein. Wild-type gD proteins from different strains or clones of HSV may also have sequence differences, e.g., having slightly different amino acid sequences. For example, in some embodiments, the gD protein is as described in UniProtKB - P03172 (GD_HHV23), UniProtKB - Q991M3 (Q991M3 _HHV1), or UniProtKB - Q69091 (GD_HHV11). In some embodiments, the gD protein as used herein comprises an amino acid sequence that has at least 80%, 85%, 90%, 95%, 98%, or 99% identity to any of SEQ ID NOs:88 and 89. In some embodiments, the fragment may consist of at least 10-20 amino acids, at least 20-30 amino acids, at least 30-50 amino acids, or the entire amino acids of the native sequence, or may be otherwise identifiable to one of ordinary skill in the art as having its origin in the native sequence.

In some embodiments, the antibody described herein comprises any suitable framework region (FR) sequences, so long as the antibody can specifically bind to an HSV-1 gD protein.

In some embodiments, the antibody described herein comprises the CDR1, CDR2, and CDR3 that comprise
(a) SEQ ID NO:17, SEQ ID NO:30, and SEQ ID NO:45, respectively;
(b) SEQ ID NO:17, SEQ ID NO:31, and SEQ ID NO:46, respectively;
(c) SEQ ID NO:18, SEQ ID NO:32, and SEQ ID NO:47, respectively;
(d) SEQ ID NO:19, SEQ ID NO:31, and SEQ ID NO:48, respectively;
(e) SEQ ID NO:20, SEQ ID NO:33, and SEQ ID NO:45, respectively;
(f) SEQ ID NO:21, SEQ ID NO:31, and SEQ ID NO:49, respectively;
(g) SEQ ID NO:18, SEQ ID NO:32, and SEQ ID NO:50, respectively;
(h) SEQ ID NO:18, SEQ ID NO:32, and SEQ ID NO:51, respectively;
(i) SEQ ID NO:22, SEQ ID NO:31, and SEQ ID NO:48, respectively;
(j) SEQ ID NO:22, SEQ ID NO:31, and SEQ ID NO:52, respectively;
(k) SEQ ID NO:23, SEQ ID NO:31, and SEQ ID NO:53, respectively;
(l) SEQ ID NO:18, SEQ ID NO:32, and SEQ ID NO:54, respectively;
(m) SEQ ID NO:17, SEQ ID NO:32, and SEQ ID NO:55, respectively;
(n) SEQ ID NO:17, SEQ ID NO:32, and SEQ ID NO:56, respectively;
(o) SEQ ID NO:22, SEQ ID NO:31, and SEQ ID NO:57, respectively;
(p) SEQ ID NO:22, SEQ ID NO:31, and SEQ ID NO:58, respectively;
(q) SEQ ID NO:18, SEQ ID NO:32, and SEQ ID NO:59, respectively;
(r) SEQ ID NO:17, SEQ ID NO:34, and SEQ ID NO:60, respectively;
(s) SEQ ID NO:24, SEQ ID NO:35, and SEQ ID NO:61, respectively;
(t) SEQ ID NO:22, SEQ ID NO:31, and SEQ ID NO:62, respectively; or
(u) SEQ ID NO:25, SEQ ID NO:36, and SEQ ID NO:63, respectively.

In some embodiments, the antibody described herein comprises a framework region 1 (FR1) selected from SEQ ID NOs:1-16, a framework region 2 (FR2) selected from SEQ ID NOs:26-29, a framework region 3 (FR3) selected from SEQ ID NOs:37-44, and a framework region 4 (FR4) selected from SEQ ID NOs:64-66.

In some embodiments, the antibody described herein comprises the FR1, FR2, FR3, and FR4 that comprise
(a) SEQ ID NO:1, SEQ ID NO:26, SEQ ID NO:37, and SEQ ID NO:64, respectively;
(b) SEQ ID NO:2, SEQ ID NO:26, SEQ ID NO:38, and SEQ ID NO:64, respectively;
(c) SEQ ID NO:3, SEQ ID NO:26, SEQ ID NO:39, and SEQ ID NO:64, respectively;
(d) SEQ ID NO:4, SEQ ID NO:26, SEQ ID NO:40, and SEQ ID NO:65, respectively;
(e) SEQ ID NO:5, SEQ ID NO:27, SEQ ID NO:37, and SEQ ID NO:64, respectively;
(f) SEQ ID NO:6, SEQ ID NO:26, SEQ ID NO:41, and SEQ ID NO:64, respectively;
(g) SEQ ID NO:7, SEQ ID NO:26, SEQ ID NO:39, and SEQ ID NO:64, respectively;
(h) SEQ ID NO:8, SEQ ID NO:26, SEQ ID NO:39, and SEQ ID NO:64, respectively;
(i) SEQ ID NO:9, SEQ ID NO:26, SEQ ID NO:40, and SEQ ID NO:65, respectively;
(j) SEQ ID NO:2, SEQ ID NO:26, SEQ ID NO:40, and SEQ ID NO:64, respectively;
(k) SEQ ID NO:10, SEQ ID NO:26, SEQ ID NO:41, and SEQ ID NO:64, respectively;
(l) SEQ ID NO:8, SEQ ID NO:26, SEQ ID NO:39, and SEQ ID NO:64, respectively;
(m)SEQ ID NO:11, SEQ ID NO:26, SEQ ID NO:38, and SEQ ID NO:64, respectively;
(n) SEQ ID NO:12, SEQ ID NO:26, SEQ ID NO:38, and SEQ ID NO:64, respectively;
(o) SEQ ID NO:13, SEQ ID NO:26, SEQ ID NO:41, and SEQ ID NO:64, respectively;
(p) SEQ ID NO:2, SEQ ID NO:26, SEQ ID NO:42, and SEQ ID NO:64, respectively;
(q) SEQ ID NO:8, SEQ ID NO:26, SEQ ID NO:39, and SEQ ID NO:64, respectively;
(r) SEQ ID NO:12, SEQ ID NO:26, SEQ ID NO:38, and SEQ ID NO:64, respectively;
(s) SEQ ID NO:14, SEQ ID NO:28, SEQ ID NO:43, and SEQ ID NO:66, respectively;
(t) SEQ ID NO:15, SEQ ID NO:26, SEQ ID NO:41, and SEQ ID NO:64, respectively; or
(u) SEQ ID NO:16, SEQ ID NO:29, SEQ ID NO:44, and SEQ ID NO:64, respectively.

The amino acid sequences of SEQ ID NOs. 1-66 are set forth below.

| | | |
|---|---|---|
| SEQ ID No: | Name | Sequence |
| SEQ ID NO: 1 | FR1-1 | QVKLEESGGGLVQPGGSLRLSCAASGF |
| SEQ ID NO: 2 | FR1-2 | QVQLVESGGGLVQPGGSLRLSCTASGF |
| SEQ ID NO: 3 | FR1-3 | HVQLVESGGGLVQPGGSLTLSCAASGF |
| SEQ ID NO: 4 | FR1-4 | QVQLVESGGGLVQPGGSLKLSCASSES |
| SEQ ID NO: 5 | FR1-5 | QVDVQLVESGGGLVQPGGSLRLSCAAS |
| SEQ ID NO: 6 | FR1-6 | QVKLEESGGGLVQPGGSLRLSCTASGF |
| SEQ ID NO: 7 | FR1-7 | EVQLVESGGGLVQPGGSLTLSCAASGF |
| SEQ ID NO: 8 | FR1-8 | QVQLVESGGGLVQPGGSLTLSCAASGF |
| SEQ ID NO: 9 | FR1-9 | QVKLEESGGGLAQPGGSLRLSCTASGF |
| SEQ ID NO: 10 | FR1-10 | DVQLVDSGGGLVQPGGSLRLSCSASGF |
| SEQ ID NO: 11 | FR1-11 | QVKLEESGGGLAQPGGSLRLSCAASGF |
| SEQ ID NO: 12 | FR1-12 | QVQLVESGGGLVQPGGSLRLSCAASGF |
| SEQ ID NO: 13 | FR1-13 | AVQLVESGGGLVQPGGSLRLSCSASGF |
| SEQ ID NO: 14 | FR1-14 | QVKLEESGGGVVQDGGSLRLSCAAI |
| SEQ ID NO: 15 | FR1-15 | QVQLVESGGGLVQPGGSLRLSCSASGF |
| SEQ ID NO: 16 | FR1-16 | DVQLVESGGGLVQPGGSLRLSCAAS |
| SEQ ID NO: 17 | CDR1-1 | SFSFKNYA |
| SEQ ID NO: 18 | CDR1-2 | SFSFENYA |
| SEQ ID NO: 19 | CDR1-3 | IFAFKNYA |
| SEQ ID NO: 20 | CDR1-4 | GFSFSFKN |
| SEQ ID NO: 21 | CDR1-5 | SFAFKDYA |
| SEQ ID NO: 22 | CDR1-6 | SFAFKNYA |
| SEQ ID NO: 23 | CDR1-7 | SSAFKNYA |
| SEQ ID NO: 24 | CDR1-8 | EQFFTTNA |
| SEQ ID NO: 25 | CDR1-9 | GFSFSFEN |
| SEQ ID NO: 26 | FR2-1 | MSWVRQAPGKGLEWVST |
| SEQ ID NO: 27 | FR2-2 | YAMTWVRQAPGKGLEWV |
| SEQ ID NO: 28 | FR2-3 | MAWFRQAPGKERELVAA |
| SEQ ID NO: 29 | FR2-4 | YAMSWVRQAPGKGLEWV |
| SEQ ID NO: 30 | CDR2-1 | MSGGGDET |
| SEQ ID NO: 31 | CDR2-2 | MSGGGGDT |
| SEQ ID NO: 32 | CDR2-3 | MSGGGGDI |
| SEQ ID NO: 33 | CDR2-4 | STMSGGGDET |
| SEQ ID NO: 34 | CDR2-5 | MSGSGGDT |
| SEQ ID NO: 35 | CDR2-6 | TDWSGQST |
| SEQ ID NO: 36 | CDR2-7 | STMSGGGGDT |
| SEQ ID NO: 37 | FR3-1 | KYADSVKGRFTISRDNTKNTLYLQMNSLKPEDTAVYYC |
| SEQ ID NO: 38 | FR3-2 | KYADSVKGRFTISRDNAKNTLYLQMNSLKPEDTAVYYC |
| SEQ ID NO: 39 | FR3-3 | KYADSVKGRFTISRDNAKNTLYLQMNNLKPEDSAVYYC |
| SEQ ID NO: 40 | FR3-4 | KYADSVKGRFTISRDNAKNTVYLQMNSLKPEDTAVYYC |
| SEQ ID NO: 41 | FR3-5 | KYADSVKGRFTISRDNNKNTVYLQMNSLKPEDTAVYYC |
| SEQ ID NO: 42 | FR3-6 | KYADSVKGRFTISRDNAKNAVYLQMNSLKPEDTAVYYC |
| SEQ ID NO: 43 | FR3-7 | SYADSVKGRFTISRDTAKNVMYLQMNILHTEDTAVYYC |
| SEQ ID NO: 44 | FR3-8 | KYADSVKGRFTISRDNAKNMLYLQMNSLKPEDTAVYYC |
| SEQ ID NO: 45 | CDR3-1 | AKGWITTDRFTNTP |
| SEQ ID NO: 46 | CDR3-2 | AKGWITTDRFTDTP |
| SEQ ID NO: 47 | CDR3-3 | AKGWITTNRFTNTP |
| SEQ ID NO: 48 | CDR3-4 | AKGWITTDQFASAP |
| SEQ ID NO: 49 | CDR3-5 | AKGWITTNQFADAP |
| SEQ ID NO: 50 | CDR3-6 | AKGWITTNRLTNTP |
| SEQ ID NO: 51 | CDR3-7 | AKGWITTNRFTNIP |
| SEQ ID NO: 52 | CDR3-8 | AEGWITTDQFASAP |
| SEQ ID NO: 53 | CDR3-9 | AKGWITNNQFASAP |
| SEQ ID NO: 54 | CDR3-10 | AKGWITTNQFTNTP |
| SEQ ID NO: 55 | CDR3-11 | AKGWIATDQFTNTP |
| SEQ ID NO: 56 | CDR3-12 | AKGWITTDQFTNTP |
| SEQ ID NO: 57 | CDR3-13 | AEGWITNNQFASAP |
| SEQ ID NO: 58 | CDR3-14 | AKGWITTDQFATAP |
| SEQ ID NO: 59 | CDR3-15 | AKGWITANRFTNTP |
| SEQ ID NO: 60 | CDR3-16 | AKGWITTDRFTDAP |
| SEQ ID NO: 61 | CDR3-17 | ARAPGRVLLTSDLESYTI |
| SEQ ID NO: 62 | CDR3-18 | AKGWITNNQFAFAP |
| SEQ ID NO: 63 | CDR3-19 | AKGWITTDRFTNTL |
| SEQ ID NO: 64 | FR4-1 | RGQGTQVTVSS |
| SEQ ID NO: 65 | FR4-2 | RGHGTQVTVSS |
| SEQ ID NO: 66 | FR4-3 | WGQGTQVTVSS |

For the purpose of this application, CDR1, CDR2, and CDR3 are located from N terminus to C terminus in the antibody described herein. FR1 is located at the N terminus of the antibody described herein, FR2 is located between CDR1 and CDR2, FR3 is located between CDR2 and CDR3, and FR4 is located at the C terminus of the antibody described herein.

In some embodiments, the antibody described herein comprises a heavy chain variable region, wherein the heavy chain variable region comprises an amino acid sequence as set forth in any one of SEQ ID NOs:67-87. In some embodiments, the antibody described herein comprises an amino acid sequence with at least 80%, 85%, 90%, 95%, 96%, 97%, 98%, 99%, or 100% identity to any one of SEQ ID NOs:67-87 (Table 3). Preferably, the sequence variation does not significantly decrease the antibody’s binding ability to a gD protein, or the sequence variation does not prevent the antibody from specifically binding to a gD protein. In some embodiments, the sequence variation does not increase the K_{D} value for the binding of the antibody described herein to a gD protein. In some embodiments, the sequence variation does not increase the K_{D} value for the binding of the antibody described herein to a gD protein by more than 1%, 5%, 10%, 15%, 20%, 25%, 30%, 35%, 40%, or 50%. In some embodiments, at least a portion of the sequence variation may occur through conservative amino acid substitution(s).

TABLE 3

| | | |
|---|---|---|
| SEQ ID NO: 67 | 3-G-4 M13R | QVKLEESGGGLVQPGGSLRLSCAASGFSFSFKNYAMSWVRQAPGKGLEWVSTMSGGGDETKYADSVKGRFTISRDNTKNTLYLQMNSLKPEDTAVYYCAKGWITTDRFTNTPRGQGTQVTVSS |
| SEQ ID NO: 68 | 3-G-6 M13R | QVQLVESGGGLVQPGGSLRLSCTASGFSFSFKNYAMSWVRQAPGKGLEWVSTMSGGGGDTKYADSVKGRFTISRDNAKNTLYLQMNSLKPEDTAVYYCAKGWITTDRFTDTPRGQGTQVTVSS |
| SEQ ID NO: 69 | 3-G-8_M13R | HVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRFTNTPRGQGTQVTVSS |
| SEQ ID NO: 70 | 3-G-10_M13R | QVQLVESGGGLVQPGGSLKLSCASSESIFAFKNYAMSWVRQAPGKGLEWVSTMSGGGGDTKYADSVKGRFTISRDNAKNTVYLQMNSLKPEDTAVYYCAKGWITTDQFASAPRGHGTQVTVSS |
| SEQ ID NO: 71 | 3-G-12 M13R | QVDVQLVESGGGLVQPGGSLRLSCAASGFSFSFKNYAMTWVRQAPGKGLEWVSTMSGGGDETKYADSVKGRFTISRDNTKNTLYLQMNSLKPEDTAVYYCAKGWITTDRFTNTPRGQGTQVTVSS |
| SEQ ID NO: 72 | 3-G-19 M13R | QVKLEESGGGLVQPGGSLRLSCTASGFSFAFKDYAMSWVRQAPGKGLEWVSTMSGGGGDTKYADSVKGRFTISRDNNKNTVYLQMNSLKPEDTAVYYCAKGWITTNQFADAPRGQGTQVTVSS |
| SEQ ID NO: 73 | 3-G-20 M13R | EVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRLTNTPRGQGTQVTVSS |
| SEQ ID NO: 74 | 3-G-22 M13R | QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNRFTNIPRGQGTQVTVSS |
| SEQ ID NO: 75 | 3-G-24 M13R | QVKLEESGGGLAQPGGSLRLSCTASGFSFAFKNYAMSWVRQAPGKGLEWVSTMSGGGGDTKYADSVKGRFTISRDNAKNTVYLQMNSLKPEDTAVYYCAKGWITTDQFASAPRGHGTQVTVSS |
| SEQ ID NO: 76 | 3-G-26 M13R | QVQLVESGGGLVQPGGSLRLSCTASGFSFAFKNYAMSWVRQAPGKGLEWVSTMSGGGGDTKYADSVKGRFTISRDNAKNTVYLQMNSLKPEDTAVYYCAEGWITTDQFASAPRGQGTQVTVSS |
| SEQ ID NO: 77 | 3-G-35 M13R | DVQLVDSGGGLVQPGGSLRLSCSASGFSSAFKNYAMSWVRQAPGKGLEWVSTMSGGGGDTKYADSVKGRFTISRDNNKNTVYLQMNSLKPEDTAVYYCAKGWITNNQFASAPRGQGTQVTVSS |
| SEQ ID NO: 78 | 3-G-36 M13R | QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITTNQFTNTPRGQGTQVTVSS |
| SEQ ID NO: 79 | 3-G-40_M13R | QVKLEESGGGLAQPGGSLRLSCAASGFSFSFKNYAMSWVRQAPGKGLEWVSTMSGGGGDIKYADSVKGRFTISRDNAKNTLYLQMNSLKPEDTAVYYCAKGWIATDQFTNTPRGQGTQVTVSS |
| SEQ ID NO: 80 | 3-G-41 M13R | QVQLVESGGGLVQPGGSLRLSCAASGFSFSFKNYAMSWVRQAPGKGLEWVSTMSGGGGDIKYADSVKGRFTISRDNAKNTLYLQMNSLKPEDTAVYYCAKGWITTDQFTNTPRGQGTQVTVSS |
| SEQ ID NO: 81 | 3-G-43 M13R | AVQLVESGGGLVQPGGSLRLSCSASGFSFAFKNYAMSWVRQAPGKGLEWVSTMSGGGGDTKYADSVKGRFTISRDNNKNTVYLQMNSLKPEDTAVYYCAEGWITNNQFASAPRGQGTQVTVSS |
| SEQ ID NO: 82 | 3-G-44_M13R | QVQLVESGGGLVQPGGSLRLSCTASGFSFAFKNYAMSWVRQAPGKGLEWVSTMSGGGGDTKYADSVKGRFTISRDNAKNAVYLQMNSLKPEDTAVYYCAKGWITTDQFATAPRGQGTQVTVSS |
| SEQ ID NO: 83 | 3-G-59 M13R | QVQLVESGGGLVQPGGSLTLSCAASGFSFSFENYAMSWVRQAPGKGLEWVSTMSGGGGDIKYADSVKGRFTISRDNAKNTLYLQMNNLKPEDSAVYYCAKGWITANRFTNTPRGQGTQVTVSS |
| SEQ ID NO: 84 | 3-G-64 M13R | QVQLVESGGGLVQPGGSLRLSCAASGFSFSFKNYAMSWVRQAPGKGLEWVSTMSGSGGDTKYADSVKGRFTISRDNAKNTLYLQMNSLKPEDTAVYYCAKGWITTDRFTDAPRGQGTQVTVSS |
| SEQ ID NO: 85 | 3-G-75 M13R | QVKLEESGGGVVQDGGSLRLSCAAIEQFFTTNAMAWFRQAPGKERELVAATDWSGQSTSYADSVKGRFTISRDTAKNVMYLQMNILHTEDTAVYYCARAPGRVLLTSDLESYTIWGQGTQVTVSS |
| SEQ ID NO: 86 | 3-G-77 M13R | QVQLVESGGGLVQPGGSLRLSCSASGFSFAFKNYAMSWVRQAPGKGLEWVSTMSGGGGDTKYADSVKGRFTISRDNNKNTVYLQMNSLKPEDTAVYYCAKGWITNNQFAFAPRGQGTQVTVSS |
| SEQ ID NO: 87 | 3-G-87 M13R | DVQLVESGGGLVQPGGSLRLSCAASGFSFSFENYAMSWVRQAPGKGLEWVSTMSGGGGDTKYADSVKGRFTISRDNAKNMLYLQMNSLKPEDTAVYYCAKGWITTDRFTNTLRGQGTQVTVSS |

In some embodiments, the antibody described herein may comprise one (single valence) or more (multiple valence) heavy chain variable regions (VHHs). In some embodiments, the antibody described herein comprises multiple, e.g., two, three, or four VHHs, which are optionally connected by one or more linker(s). In some embodiments, the antibody described herein comprises two VHHs, which are optionally connected by a linker. In some embodiments, each of the two VHHs has a sequence as set forth in any one of SEQ ID NO: 67-87.

In some embodiments, the antibody described herein is a multispecific antibody, e.g., a bispecific antibody, that comprises one or more VHHs disclosed here. For example, the multispecific antibody may comprise a VHH selected from the sequences set forth in SEQ ID NOs:67-87, and comprise another binding moiety (e.g., a variable region) that binds another epitope or antigen. As another example, the multispecific antibody may comprise two or more VHHs selected from the sequences set forth in SEQ ID NOs:67-87, and comprise another binding moiety (e.g., a variable region) that binds another epitope or antigen, wherein the two or more VHHs may be the same or different, optionally linked by one or more linkers. In some embodiments, the multi-specific antibody comprises an amino acid sequence set forth in SEQ ID NO:85 and one or more amino acid sequences selected from SEQ ID NOs:67-84 and 86-87.

In some embodiments, the gD protein that the antibody described herein specifically binds to is derived from a virus. In some embodiments, the virus is any virus that expresses gD protein. In some embodiments, the virus is an HSV. In some embodiments, the HSV is an HSV-1 or an HSV-2.

As used herein, the term “gD protein derived from HSV,” “HSV derived gD protein,” or “HSV gD protein” refers to the origin or source of the gD protein being HSV, which may include wild-type HSV gD proteins, recombinant HSV gD proteins, synthesized HSV gD proteins, purified HSV gD proteins, isolated HSV gD proteins, HSV gD proteins that are fused with another protein, HSV gD proteins that are conjugated with a label, or a fragment thereof. In some embodiments, the fragment comprises the ectodomain of an HSV gD protein or a portion thereof.

In some embodiments, the gD protein that the antibody described herein specifically bind to is a recombinant gD protein. As used herein, a recombinant protein refers to a protein that is encoded by a recombinant DNA. A recombinant DNA is a piece of DNA that is created by combining at least two fragments from two different sources. Modification of the gene by recombinant DNA technology can lead to expression of a mutant protein. Recombinant protein is obtained by cloning the recombinant DNA encoding the protein into a system that supports gene expression and RNA translation, i.e., an expression system. It’s understood that a skilled artisan is able to select a suitable expression system and conditions for recombinant DNA expression. Some non-limiting examples of expression system are mammalian expression system, bacterial expression system, yeast expression system, and insect expression system. In some embodiments, the recombinant HSV-1 gD protein comprises an HSV-1 gD and a Fc region or a fragment of Fc.

In some embodiments, the antibody described herein binds HSV-1 and/or HSV-2. In some embodiments, the antibody described herein binds HSV-1 and/or HSV-2 by targeting the gD protein on the surface of HSV-1 and/or HSV-2.

In some embodiments, the antibody described herein binds the gD protein with a K_{D} ranging from about 1 pM to about 1 µM. In some embodiments, the K_{D} ranges from about 50 pM to about 4 nM. In some embodiments, the K_{D} ranges from about 0.05 nM to 0.2 nM. In some embodiments, the K_{D} is about 4 nM or lower.

K_{D} refers to a dissociation rate of the antibody/antigen interaction. Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody] [antigen] antibody/antigen complex formation is equal to the rate of dissociation into its components. K_{D} and affinity are inversely proportional, so the smaller the K_{D} value the greater the affinity of the antibody for its target. The affinity determination of monoclonal antibodies can perform high-precision detection because they are only selective for the same epitope. But in the case of polyclonal antibodies, only the average affinity can be obtained because they detect epitopes that are heterogeneous and have different antibody/antigen complex mixture. For a monoclonal antibody, the K_{D} is calculated with the formula: K_{D}=[antibody][antigen]/[antibody/antigen complex], wherein [X] refers to the concentration of X. Methods to determine antibody affinity include, but not limited to, ELISA-based methods, as well as other biophysical methods, such as micro-scale thermophoresis (MST) and surface plasmon resonance (SPR). Preferably, SPR is used to measure the K_{D} of the binding between the antibody described herein and a gD protein. More information about SPR and its protocol can be found in Surface Plasmon Resonance, Nico J. MolMarcel J. E. Fischer, DOI:10.1007/978-1-60761-670-2 ISBN: 978-1-60761-669-6.

In some embodiments, the multiple antibodies described herein bind to different epitopes on the gD protein, thus in some embodiments, they may have different effects on the biology and function of this glycoprotein in the infectivity of HSV-1 and/or HSV-2.

In some embodiments, the antibody described herein is a single domain antibody (sdAb). In some embodiments, the antibody described herein is a single heavy domain antibody. As used herein, “single domain antibody” (sdAb) refers to an antibody consisting of a single variable domain of an IgG antibody. For example, in some embodiments, the single domain antibody is a single heavy chain domain antibody, or a VHH antibody, which consists of a single variable domain of heavy chain of a heavy chain IgG (hcIgG) molecule produced by a Camelidae family mammal (e.g., llamas, camels, and alpacas).

In some embodiments, the antibody described herein is a neutralizing antibody. In some embodiments, the antibody described herein is capable of neutralizing HSV-1 and/or HSV-2. In some embodiments, the neutralizing antibody has an amino acid sequence as set forth in SEQ ID NO:85. In some embodiments, the neutralizing antibody has an amino acid sequence with at least 90%, 95%, 96%, 97%, 98%, 99%, or 100% identity to SEQ ID NO:85. Preferably, the sequence variation does not significantly decrease the antibody’s binding ability to a gD protein, or the sequence variation does not prevent the antibody from specifically binding to a gD protein. In some embodiments, the sequence variation does not increase the K_{D} value for the binding of the antibody described herein to a gD protein. In some embodiments, the sequence variation does not increase the K_{D} value of the binding between the antibody described herein and a gD protein by more than 1%, 5%, 10%, 15%, 20%, 25%, 30%, 35%, 40%, or 50%. In some embodiments, at least a portion of the sequence variation may occur through conservative amino acid substitution(s).

A neutralizing antibody (NAb) is an antibody that defends a cell from a pathogen or infectious particle (e.g., HSV-1 or HSV-2) by neutralizing any effect it has biologically. Neutralization renders the particle no longer infectious or pathogenic. Neutralizing antibodies can inhibit the infectivity by binding to the pathogen and block the molecules needed for cell entry. In some embodiments, neutralizing antibodies can neutralize the biological effects of the antigen without a need for immune cells. Neutralization assays are capable of being performed and measured in different ways, including but not limited to the use of techniques such as plaque reduction which compares counts of virus plaques in control wells with those in inoculated cultures, microneutralization which is performed in microtiter plates filled with small amounts of sera, or colorimetric assays which depend on biomarkers indicating metabolic inhibition of the virus (Kaslow, R. A.; Stanberry, L.R.; Le Duc, J. W., eds. (2014). Viral Infections of Humans: Epidemiology and Control (5th ed.). Springer. p. 56. ISBN 9781489974488).

In some embodiments the antibody described herein is a non-neutralizing antibody. Non-neutralizing antibodies bind specifically to the pathogen, but do not interfere with their infectivity. That might be because they do not bind to the right region. Non-neutralizing antibodies can be important to flag the particle for immune cells, signaling that it has been targeted, after which the particle is processed and consequently destroyed by recruited immune cells.

In some embodiments, the antibody described herein is humanized or partially humanized. As used herein, “humanized” or “humanization” means that the amino acid sequence of the antibody described herein is modified to reduce its immunogenicity in human. Humanization is usually achieved by modifying the antibody’s sequence to increase its similarity to its counterpart produced naturally in humans. Two major approaches have been used to transform murine antibodies into humanized antibodies: rational design and empirical methods. The rational design methods are characterized by antibody structural modeling, generating a few variants of the engineered antibodies and assessing their binding or any other property of interest. If the designed variants do not produce the expected results, a new cycle of design and binding assessment is initiated. The rational design methods include, but are not limited to, complementarity determining region (CDR) grafting, resurfacing, super-humanization and human string content optimization, among which, CDR grafting is the most widely used. Humanized antibody generated by CDR-grafting contains amino acids from the six CDRs of the parental murine mAb, which are grafted onto a human antibody framework. The low content of non-human sequence in humanized antibodies (^{~}5%) has proven effective in both reducing the immunogenicity and prolonging the serum half-life in humans (7).

Simple grafting of CDR sequences often yields humanized antibodies that bind antigen much more weakly than the parental murine mAb, and decreases in affinity of up to several hundred-fold have been reported (Eigenbrot et al., 1994, Proteins 18, 49-62). To restore high affinity, the antibody must be further engineered to fine tune the structure of the antigen-binding loops. This is usually achieved by replacing key residues in the framework regions of the antibody variable domains with the matching sequence from the parental murine antibody. These framework residues are usually involved in supporting the conformation of the CDR loops, although some framework residues may themselves directly contact the antigen (Mian et al., 1991, J Mol Biol 217, 133-151). It has become apparent that the accomplishment of antibody humanization by rational method faces relatively high uncertainty. Moreover, broad application of this technology has also been restricted due to reliance on structural biology, which is not readily available for many laboratories.

In contrast to the rational design methods, empirical methods do not require the structure information of the antibody. They depend on the generation of large combinatorial libraries and selection of the desired variants by enrichment technologies such as phage, ribosome or yeast display, or by high throughput screening techniques. These methods rest on selection rather than making assumptions on the impact of mutations on the antibody structure. These methods include, but are not limited to, framework libraries, guided selection, framework shuffling and humaneering. However, the success of these methods relies mainly on the construction of large libraries, because high affinity antibodies can be isolated from the large size of antibody repertoires.

In some embodiments, the antibody described herein is conjugated with a label or a drug moiety such as a toxin.

### Compositions

In another aspect, the present disclosure provides a composition comprising the antibody described herein.

In another aspect, the present disclosure provides a pharmaceutical composition comprising the antibody described herein and a pharmaceutically acceptable carrier.

The composition or the pharmaceutical composition described herein may comprise a pharmaceutically acceptable carrier, diluent, or excipient. As used herein “pharmaceutically acceptable carrier, diluent, or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose, and sucrose; starches, such as corn starch and potato starch; cellulose, and its derivatives, such as sodium carboxymethyl cellulose, ethyl cellulose, and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate, and ethyl laurate; agar; buffering agents, such as magnesium hydroxide and aluminum hydroxide; alginic acid; pyrogen-free water; isotonic saline; Ringer’s solution; ethyl alcohol; phosphate buffer solutions; and any other compatible substances employed in pharmaceutical formulations.

The liquid pharmaceutical compositions, whether they be solutions, suspensions or other like form, may include one or more of the following: sterile diluents such as water for injection, saline solution, preferably physiological saline; Ringers solution; isotonic sodium chloride; fixed oils such as synthetic mono or diglycerides which may serve as the solvent or suspending medium; polyethylene glycols; glycerin; propylene glycol or other solvents; antibacterial agents, such as benzyl alcohol or methyl paraben; antioxidants such as ascorbic acid or sodium bisulfite; chelating agents, such as ethylenediaminetetraacetic acid; buffers such as acetates, citrates, or phosphates; and agents for the adjustment of tonicity, such as sodium chloride or dextrose. The parenteral preparation can be enclosed in ampoules, disposable syringes, or multiple dose vials made of glass or plastic. An injectable pharmaceutical composition is preferably sterile.

The composition may be suitably developed for intravenous, intratumoral, oral, rectal, vaginal, parenteral, topical, pulmonary, intranasal, buccal, ophthalmic, or another route of administration.

### Polynucleotides

In another aspect, the present disclosure provides a polynucleotide that encodes the antibody described herein. The polynucleotide sequences encoding the antibody described herein can be operably linked to one or more regulatory elements, such as a promoter and enhancer, that allow expression of the nucleotide sequence in the intended host cell. The polynucleotide may be a cDNA. The polynucleotide described herein is obtained by methods readily available in the arts.

### Vectors

In another aspect, the present disclosure provides a vector comprising the polynucleotide as described herein. Such vectors may be plasmid vectors, viral vectors, vectors for baculovirus expression, transposon-based vectors, or any other vector suitable for introduction of the polynucleotide of the disclosure into a given organism or genetic background by any means. For example, polynucleotides encoding the antibody described herein may be inserted into expression vectors. The DNA segments encoding the antibody may be operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells, or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian, or other animal origins.

### Cells

In another aspect, the present disclosure provides a cell capable of expressing the antibody described herein. In another aspect, the present disclosure provides a cell comprising the polynucleotide described herein or the vector described herein.

The cell described herein include, but not limited to, eukaryotic cells, prokaryotic cells, plant cells, or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian, or other animal origins.

### Methods

The present disclosure also provides methods for producing and using the antibodies described herein.

In another aspect, the present disclosure provides a method of producing an antibody, comprising culturing the cell described herein and recovering the antibody from the cell.

The antibody provided herein can be produced by any method known to those of skill in the art including in vivo and in vitro methods. Desired antibody can be expressed in any organism suitable to produce the required amounts and forms of the antibody. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modification that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Expression vectors can be introduced into host cells via, for example, transformation, transfection, transduction, infection, electroporation, and sonoporation. A skilled artisan is able to select methods and conditions suitable for introducing an expression vector into host cells.

Following the introduction of a vector comprising a selectable marker, cells can be allowed to grow for 1-2 days in an enriched media before they are switched to selective media. The purpose of the selectable marker is to confer resistance to selection, and its presence allows growth and recovery of cells that successfully express the introduced sequences. Resistant cells of stably transformed cells can be proliferated using tissue culture techniques appropriate to the cell types. In some embodiments, the antibody described herein is expressed in a mammalian expression system. Expression constructs can be transferred to mammalian cells by viral infection, such as by adenovirus constructs, or by direct DNA transfer, such as liposomes, calcium phosphate, DEAE-dextran, and by physical means such as electroporation and microinjection. In some embodiments, the antibody described herein is delivered using viral transduction, for example, with a vector.

Expression vectors for mammalian cells typically include an mRNA cap site, a TATA box, a translational initiation sequence (Kozak consensus sequence), and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene.

Once the vector has been incorporated into the appropriate host cell, the host cell is maintained under conditions suitable for expression of the antibody encoded by the incorporated polynucleotides. A skilled artisan is able to select conditions suitable for expression of the antibody described herein.

In another aspect, the present disclosure provides a method for treating infection of HSV-1 and/or HSV-2 in a subject, comprising administering to the subject an effective amount of the antibody as described herein or the composition as described herein. In some embodiments, the method described herein is used to treat diseases caused by HSV-1 and/or HSV-2 infection, including but not limited to oral herpes and genital herpes.

Oral herpes infection is mostly asymptomatic, and most people with HSV-1 infection are unaware they are infected. Symptoms of oral herpes include painful blisters or open sores called ulcers in or around the mouth. Sores on the lips are commonly referred to as “cold sores.” Infected persons will often experience a tingling, itching or burning sensation around their mouth, before the appearance of sores. After initial infection, the blisters or ulcers can periodically recur. The frequency of recurrences varies from person to person.

Genital herpes caused by HSV-1 and/or HSV-2 can be asymptomatic or can have mild symptoms that go unrecognized. When symptoms do occur, genital herpes is characterized by one or more genital or anal blisters or ulcers. After an initial genital herpes episode, which can be severe, symptoms may recur.

In another aspect, the present disclosure provides a method for detecting HSV-1 and/or HSV-2 in a sample, comprising contacting the sample with the antibody described herein or the composition described herein, and detecting the presence of the antibody. Methods for detection of antibodies include but not limited to immunoprecipitation assay, in which Ag-Ab complex aggregates are detected, often by hemagglutination; immunocytochemistry, for in situ Ab detection in tissue slices; immunoblotting (dot blot technique) whereby Ag-Ab aggregates are trapped on membranes and then detected with a secondary Ab to yield spots; and immunosorbent assays, which are similar to immunoblotting but, by using a tagged secondary antibody, allow the primary antibody to be quantified. A variety of immunosorbent kits are available which permit rapid, specific, accurate and sensitive detection particularly of IgE antibodies. It is understood that a skilled artisan is able to select the suitable method and condition to carry out the antibody detection. More information regarding antibody detection can be found in Hermann K., Ollert M., Ring J. (2005) Antibody detection. In: Nijkamp F.P., Parnham M.J. (eds) Principles of Immunopharmacology. Birkhäuser Basel. https://doi.org/10.1007/3-7643-7408-X_11.

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1 Production and Inactivation of HSV-1

HSV-1 virus was produced at Virogin Biotech using Vero cells and was purified and concentrated using high speed centrifugation as described in Bernstock et al.

The virus was inactivated by exposure to 27 mJ/cm² of UVc by placing 1 ml of purified virus in a 10 cm uncovered petri dish. Inactivation was verified by performing a plaque assay on the virus using Vero cells.

### Example 2 Expression and Purification of HSV-1 gD Protein

HSV-1 gD protein coding sequence was cloned into pCDNA3.1 vector, and expression was performed using FreeStyle 293 cells expression system (Thermofisher). Briefly, the FreeStyle 293 cells were grown in FreeStyle 293 Expression Medium (Thermofisher) until cells reached the density of 1 million cell/ml. The cells were transfected with the expression plasmid (pCDNA3.1-gD-Fc) using a polyethylenimine-based method (PEI) as described in Baldi et al. Medium was harvested 3 days after transfection, and recombinant protein was purified on protein G column as described in Agrawal et al. Briefly, the medium was filtered using 0.45 µm filter and loaded in protein A column. The column was washed with 5 CV of PBS, then eluted using citrate buffer, pH 3.2.

### Example 3 Immunization in Llama

One llama (Lama glama) was immunized with inactivated HSV-1 at Cedarlane Laboratories (Burlington, Canada). The immunization consisted of a prime injection of 100 µg of HSV-1 (100 µL of a 1 mg/mL stock) mixed with Complete Freund’s Adjuvant on Day 0, followed by three boosts on Days 21, 28 and 35 with 100 µg of HSV-1 mixed with Incomplete Freund’s Adjuvant in each boost. Before the first injection on Day 0, pre-immune blood was drawn and the resulting serum frozen. On Day 42 immune blood was drawn and the resulting serum frozen. PBMCs were also isolated from blood drawn on Day 42 and frozen immediately.

### Example 4 Detection of Antibodies of HSV-1 and HSV-1 gD Protein

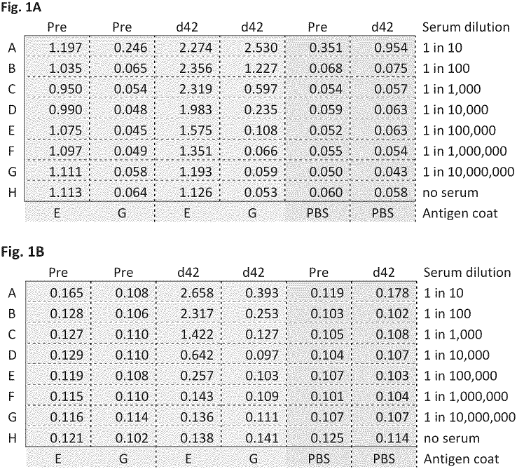

Using pre-immune serum drawn before the prime injection on Day 0 and immune serum drawn on Day 42 post immunization, ELISA was used to determine if a polyclonal response was generated to HSV-1 and glycoprotein D. Polyclonal llama antibodies specific to the targets were detected with: (i) an anti-llama-Fc-IgG HRP conjugate for detection of both conventional and heavy-chain IgGs, and (ii) an anti-heavy-chain IgG specific mouse mAb (called “1C10”), for detection of heavy-chain IgGs (Henry et al., 2019). ELISA plate wells were coated overnight at 4° C. with 0.1 µg of HSV-1 or glycoprotein D per well diluted in PBS (100 µL/well). The next day wells were blocked with 4% w/v non-fat skimmed milk diluted in PBS (300 µL/well) for 1 h at 37° C. After removal of blocking buffer, serum from Days 0 and 42 was added to wells in a 10-fold dilution series (starting at 1:10 to 1:10,000,000) diluted in PBS (100 µL/well) for 1 h at room temperature. Wells were washed 3× with PBS-T (PBS, 0.05% v/v Tween 20), 300 µL/well per wash. Next, for total llama polyclonal detection, an anti-llama-Fc IgG HRP conjugate (Bethyl Laboratories, Montgomery, TX) diluted 1:20,000 in PBS was added to each well (100 µL/well) for 1 h at room temperature. Alternatively, for heavy-chain IgG detection, wells were incubated with mouse 1C10 mAb (NRC, Ottawa, Canada) diluted 1:1,000 in PBS (100 µL/well) for 1 h at room temperature. All wells were washed again as above. The total llama polyclonal wells were developed by adding TMB peroxidase substrate (Mandel Scientific, Guelph, Canada) for 5 mins (100 µL/well) at room temperature and the reaction stopped with 1 M sulfuric acid (100 µL/well) and absorbance read at 450 nm. To the heavy-chain polyclonal wells, after washing as above, a donkey-anti-mouse IgG HRP conjugate diluted 1:3,000 in PBS was added (100 µL/well) for 1 h at room temperature. Wells were again washed and developed with TMB substrate, stopped and absorbance read as above. A total polyclonal response and a heavy-chain IgG polyclonal response to HSV-1 and glycoprotein D was observed in Day 42 serum (FIG. 1). The HSV-1 signal was high for both pre-immune and immune serum using the anti-llama-Fc IgG HRP antibody, however, the pre-immune signal was negligible when using the 1C10 mAb for detection.

### Example 5 Construction of Phage Display Library

Using PBMCs isolated from blood drawn on Day 42, a phage display library was constructed in the pMED 1 phagemid vector exactly as described in Baral et al. and Hussack et al.. Briefly, RNA was extracted from PBMCs, cDNA was synthesized and two rounds of PCR were performed to amplify VHH-encoding genes. Next, SfiI-digested PCR products were ligated into SfiI-digested pMED 1 vector and electroporated into electrocompetent TG1 E. coli cells to produce the library cells. Phage were rescued using M13KO7 helper phage (New England Biolabs, Ipswich, MA), purified, titered and used as the input for round 1 of panning. Random colonies were subjected to colony PCR as described in Baral et al.to confirm VHH inserts. PCR amplicons were Sanger DNA sequenced to assess library diversity. A library size of approximately 2 × 10⁷ unique transformants was determined.

### Example 6 Panning

Using purified library phage and glycoprotein D coated on microtitre plate wells, three rounds of phage panning were performed to isolated target specific VHHs essentially as described in Baral et al. and Hussack et al. Before round 1, wells were coated overnight at 4° C. with either PBS or 5 µg of glycoprotein D diluted in PBS (100 µL/well). The next morning, using single colonies from an M9 minimal media plate, TG1 E. coli cultures were grown in 3 mL of 2YT + 2% v/v glucose (300 µL of 20% sterile filtered glucose), no antibiotics, in a 15 mL Falcon tube at 37° C., 250 rpm, until an OD600 = 0.5 (approximately 3 h). The contents of the overnight coated wells were then removed, 2% w/v non-fat skimmed milk (NFSM) diluted in PBS (300 µL/well) added as a blocking agent, and incubated for 2 h at 37° C. After removing the blocking agent, 50 µL of library phage (a total of 3 × 1011 cfu) was added to both wells with 50 µL of 4% w/v NFSM diluted in PBS and incubated at room temperature for 30 min. After 30 mins the phage were removed and discarded. Both wells were washed with 5 × 300 µL/well PBST (PBS + 0.05% v/v Tween 20) and then 5 × 300 µL/well PBS. Bound phage were eluted after the final wash by high pH and low pH elution. First, 100 µL of freshly made triethylamine (TEA) was added to wells, incubated for 10 min, removed, transferred to a 1.5 mL Eppendorf tube, and then neutralized with 50 µL of 1 M Tris-HCl, pH 7.4. Second, 100 µL of 100 mM glycine, pH 2, was added to wells, incubated for 10 min, removed, transferred to a new 1.5 mL Eppendor tube, and then neutralized with 10 µL of 2 M Tris base. Both elutions were pooled, given a final volume of 260 µL. Using the TG1 E. coli cells started earlier, half of the phage elution (130 µL) was added to the 3 mL culture and incubated at 37° C. for 30 min without shaking. An aliquot of infected TG1 E. coli cells was then serially diluted on 2YT + ampicillin plates and grown overnight at 32° C. These plates were used to determine the eluted phage titer and were a source of colonies for colony PCR, sequencing and phage ELISA (in later panning rounds). To the remaining 3 mL culture, incubate an additional 30 min at 37° C. with 250 rpm of shaking. After 30 min of shaking, 3 µL of stock ampicillin (100 mg/mL) and M13KO7 helper phage (20 × excess phage, approximately 5 × 1010 pfu) were added, incubated for 15 min at 37° C. without shaking, before centrifugation of cells (4,000 rpm, 10 min) and supernatant discarded. The cell pellet was then resuspended in 10 mL of 2YT + ampicillin + 0.1% v/v glucose in a 50 mL Falcon tube, grown for 30 min at 37° C. with 250 rpm shaking. Next, 10 µL of stock kanamycin (50 mg/mL) was added and the culture grown overnight at 32° C. and 250 rpm shaking. The next day amplified phage particles were purified using standard PEG precipitation (25% w/v PEG, 2.5 M NaCI). Purified phage were finally resuspended in 200 µL of PBS and titers determined by absorbance measurements and titering TG1 E. coli. The amplified phage served as input for the second round of panning. Round 2 and round 3 of panning were performed exactly as described above, with the exception of SuperBlock (Thermo Fisher, Ottawa, Canada) used instead of NFSM as a blocking agent in round 2. Colonies from round 3 eluted phage titer plates (2YT + ampicillin plates; not the amplified phage) were used for phage ELISA, colony PCR and Sanger DNA sequencing.

### Example 7 Phage ELISA

Before performing phage ELISA, glycerol stocks were prepared. Using 93 colonies from round 3 eluted phage titer plates, individual colonies were picked and added to 2YT + ampicillin in 96 well sterile plates (100 µL/well), grown at 37° C. with shaking (200 rpm) for 3-4 h, and 5 µL aliquots of culture removed to a fresh plate containing 2YT + ampicillin (200 µL/well) in a replica plate. Glycerol (25 % final) was added to the original plate and frozen at -80° C., to create the glycerol stock in 96-well plate format. The second plate (containing ~200 µL/well cultures) was grown at 37° C., with 200 rpm shaking for approximately 3-4 h until an OD600 = 0.5 and M13KO7 helper phage added to each well (20 × excess phage). The plate was incubated at 37° C. for 15 min without shaking, followed by 30 min of shaking at 230 rpm. Next, 2 µL of kanamycin stock was added to each well and the plate grown overnight at 37° C. with 230 rpm shaking. The next day the plate was centrifuged to pellet the cultures and the supernatant (containing the phage) was removed and used directly for phage ELISA.

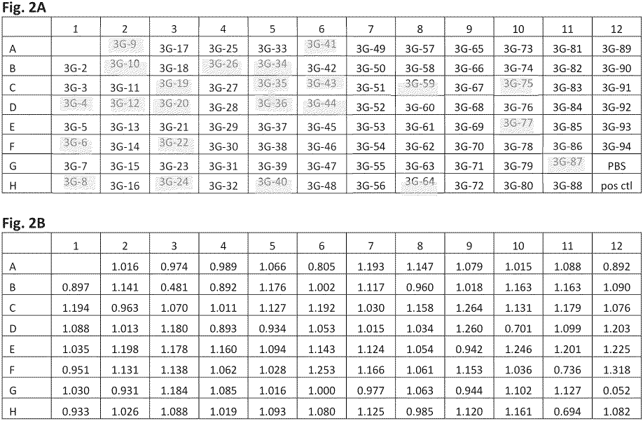

For phage ELISA, 96-well plates were coated overnight at 4° C. with 0.5 µg of glycoprotein D diluted in PBS (100 µL/well). The next day wells were blocked with 5% NFSM diluted in PBST for 1 h at 37° C. After removing the blocker, 150 µL of the phage supernatant prepared earlier was added to wells. PBS alone and pure helper phage served as assay controls. Phage were incubated for 1 h at 37° C., before 4× washes with PBST (300 µL/well). Phage binding to the surface-coated glycoprotein D were detected with an anti-M13-IgG HRP conjugate (Cytiva, Vancouver, Canada) diluted 1:5,000 in PBS and incubated for 1 h at room temperature. A final set of 4 washes was performed before addition of TMB substrate (Mandel Scientific) for 5 min (100 µL/well), then the reaction was stopped with 1 M sulfuric acid (100 µL/well) before reading the absorbance at 450 nm. FIG. 2 shows the 96 well plate layout, VHH clone names, and the resulting A450 values.

### Example 8 DNA Sequencing

Single TG1 E. coli colonies used for phage ELISAs were PCR amplified and subjected to Sanger DNA sequencing. Colony PCR was performed using primers described in Baral et al. and a standard PCR reaction mixture in a 10 µL volume. Colonies were touched with small pipette tips and added to the PCR reaction mixture, before a PCR program was run. The PCR program comprises: 95° C. for 5 min, 35 cycles of 95° C. (20 s), 55° C. (20 s) and 72° C. (20 s), before a final 5 min at 72° C. PCR products were analyzed by DNA agarose gel electrophoresis and then sequenced using the M13RP sequencing primer described in Baral et al.. Sequences were analyzed and grouped based on CDR3s. The antibody whose name is highlighted in FIG. 2A has an amino acid sequence as listed in Table 3.

### Example 9 Expression and Purification of Soluble VHH

The unique VHH genes of the clones that were scored positive in phage ELISA were sub-cloned into the expression vector (pET22b vector). After sequence confirmation, recombinant sdAbs were expressed and purified by IMAC. Briefly, the clones were inoculated in 25 ml of LB with 100 µg per ml-ampicillin and incubated at 37° C. overnight while being shaken at 200 rpm. Twenty milliliters of the culture was then added to 1 L of M9 medium comprising 0.2% glucose, 0.6% Na2HPO4, 0.3% KH2PO4, 0.1% NH4Cl, 0.05% NaCl, 1 mM MgCl2, and 0.1 mM CaCl2. This medium had also been supplemented with 0.4% casaminoacids, 5 µg/ml vitamin B1 and 100 µg/ml ampicillin. The suspension was then incubated for 24 h. One hundred milliliters of 10× TB nutrients comprising 12% Tryptone, 24% yeast extract and 4% glycerol along with 2 ml of 100 µg/ml ampicillin and 1 ml of 1 M isopropyl-beta-D-thiogalactopyranoside (IPTG) was added to the culture. The cells were further incubated for 65-70 h at 28° C. while being shaken at 200 rpm. The suspensions were then centrifuged and the cell pellets were lysed subsequently by using lysozyme. The cell lysates were centrifuged and the resulting supernatants were loaded on to 5 ml HiTrap™chelating HP affinity columns (GE Healthcare). His-tagged proteins were eluted with a linear gradient of 2.5 to 500 mM immidazole after washing the columns with four column volumes of wash solution comprising 10 mM HEPES containing 500 mM NaCl and 20 mM immidazole at pH 7.5. The eluted proteins were then dialyzed in PBS buffer.

### Example 10 Size Exclusion Chromatography (SEC) and Surface Plasmon Resonance (SPR) Analysis

Purified glycoprotein D-specific VHHs were further SEC purified to remove trace aggregates prior to SPR by injecting 300-500 µg VHH in 500 µL over a Superdex S75 10/300 GL column (Cytiva) at a flow rate of 0.8 mL/min at room temperature. HBS-EP+ (10 mM HEPES, 150 mM NaCl, 3 mM EDTA, 0.05% P20 surfactant, pH 7.4; Cytiva) was used as the running buffer. SEC fractions (0.5 mL) were collected and monomer peak fractions measured by NanoDrop to determine concentrations prior to SPR. Specific instrument, materials and methods for SEC are listed below:
Instrument: AKTÄ FPLC (GE Healthcare)
Column: Superdex S75 10/300 GL, Code: 17-5174-01, Lot: 10221448, ID 0110 (GE Healthcare)
Column: Superdex S200 INCREASE 10/300 GL, Code: 28-9909-44, Lot: 10243519, ID 0150 (GE Healthcare)
Running buffer: HBS-EP+ (see above)
Pump speed: 0.8 mL/min, sample volume: variable, fraction volume: 500 uL

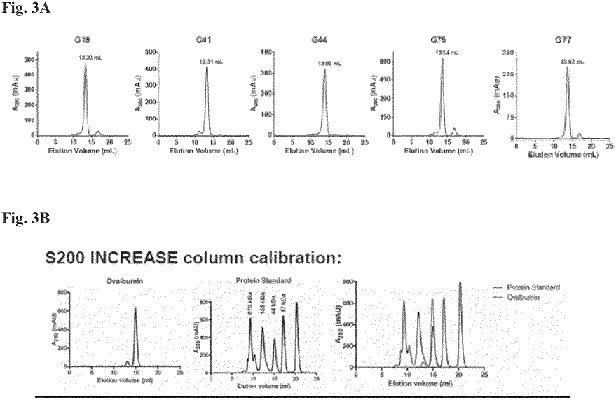

Monomer peaks for all 5 VHHs were isolated (FIG. 3A, G19 = 3-G-19_M13R, G41 = 3-G-41_M13R, G44= 3-G-44_M13R, G77 = 3-G-77_M13R, G75 = 3-G-75_M13R). Purity check of G-Fc was performed on S200 INCREASE columns (FIG. 3B). Certain information of G-Fc protein, VHH, and goat anti-human IgG are in Table 4.

TABLE 4

| | M.W. (Da) | | Ext. coeff. (M^{-l}cm⁻¹) | pI | Conc. (mg/mL) | SEC fraction | OD₂₈₀ | Conc. µM |
|---|---|---|---|---|---|---|---|---|
| G-Fc | 63237.7 | | 87500 | 5.6 | 1.62 | | | |
| G19 | 16396.1 | | 28545 | 8.89 | 1.75 | 26 | 0.205 | 7.2 |
| G41 | 16394.1 | | 28545 | 9.15 | 1.73 | 26 | 0.178 | 6.2 |
| G44 | 16402.1 | | 30035 | 8.96 | 2.04 | 27 | 0.207 | 6.9 |
| G75 | 16873.6 | | 28545 | 6.39 | 4.54 | 26 | 0.446 | 15.6 |
| G77 | 16396.1 | | 28545 | 9.33 | 2.41 | 26 | 0.209 | 7.3 |
| Reagent | | Source (supplier, Cat#) | | | M.W. (Da) | Conc. | Buffer | |
| Goat anti-human IgG | | Jackson Immuno Research, Cat#: 109-005-098, Lot: 131913 | | 150000 | | 1.3 mg/mL | | 0.01 M sodium phosphate, 0.25 M NaCl, pH 7.6 |

Various VHHs were analyzed by SPR against target G-Fc under the following conditions:
Instrument: BIACORE T200
Running buffer: HBS-EP+
Flow path: Fc 1 - 4, Detection: Fc 2-1, 3-1 and 4-1
Capture of targets B-Fc and G-Fc on Fc 2, 3 & 4: flow rate: 10 AL/min, injection time: variable (90 - 135 s),
concentration: B-Fc at 5 Ag/mL and G-Fc at 1 Ag/mL
Injection of SEC purified sdAbs on Fc 1-4: flow rate: 40 AL/min
Injection volume: 120 uL (180 s), dissociation: B-Fc 600 s, G-Fc 900 s
Biacore kinetics method: Single Cycle Kinetics (SCK)
Regeneration: flow rate: 30 uL/min, 60 uL (120 s) 10 mM glycine, pH 1.5

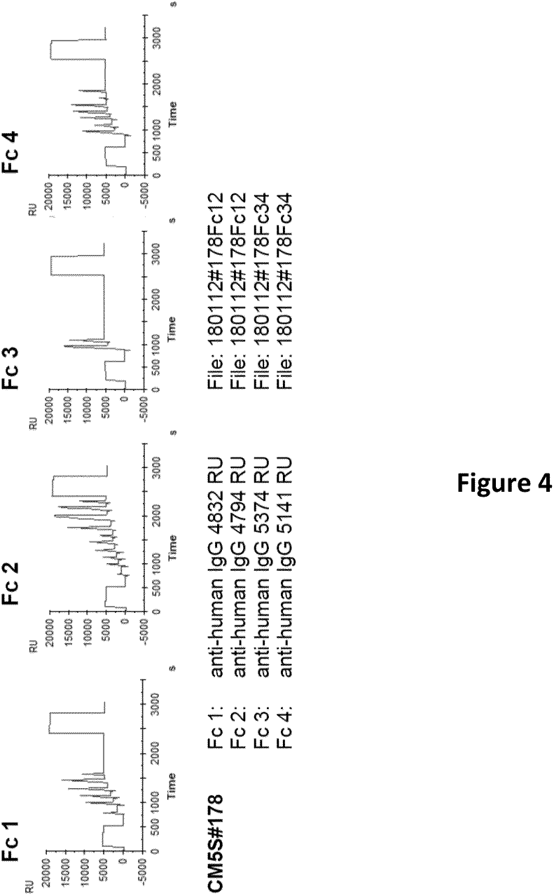

More specifically, SPR experiments were performed on a Biacore T200 (Cytiva) at 25° C. in HBS-EP+ running buffer. To determine VHH affinities and kinetics, G-Fc was captured on an anti-human Fc IgG surface and VHHs were flowed over. Approximately 5000 RUs of goat-anti-human IgG (Jackson ImmunoResearch, West Grove, PA) were amine coupled to a Series S Sensor Chip CM5 (Cytiva) using 10 mM acetate buffer, pH 4.5, following the manufacturer’s instructions. The G-Fc protein was then captured on the goat-anti-human IgG surface by injecting G-Fc (1 µg/mL) at a flow rate of 10 µL/min for 135 s, resulting in G-Fc capture levels ranging from 229 - 337 RUs. A flow cell without the G-Fc antigen captured served as a reference surface. A flow cell with a control Fc-fused protein served as a negative control surface (FIG. 4).

VHHs were injected at various concentrations ranging from 2 - 0.125 nM to 128 - 8 nM, depending on the VHH, using single cycle kinetics at a flow rate of 40 µL/min, with 180 s of contact time and 900 s of dissociation time. Injection concentration for each VHH is listed in Table 5.

TABLE 5

| | |
|---|---|
| sdAb | Concentrations |
| G19 | 8, 16, 32, 64 & 128 nM |
| G41 | 0.125, 0.25, 0.5, 1 & 2 nM |
| G44 | 0.125, 0.25, 0.5, 1 & 2 nM |
| G75 | 2, 4, 8, 16 & 32 nM |
| G77 | 0.125, 0.25, 0.5, 1 & 2 nM |

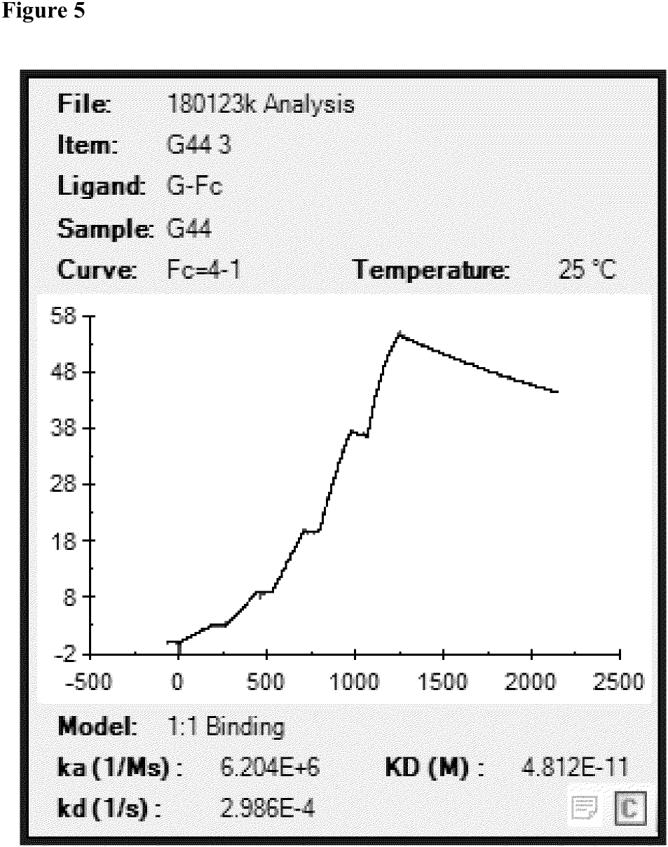

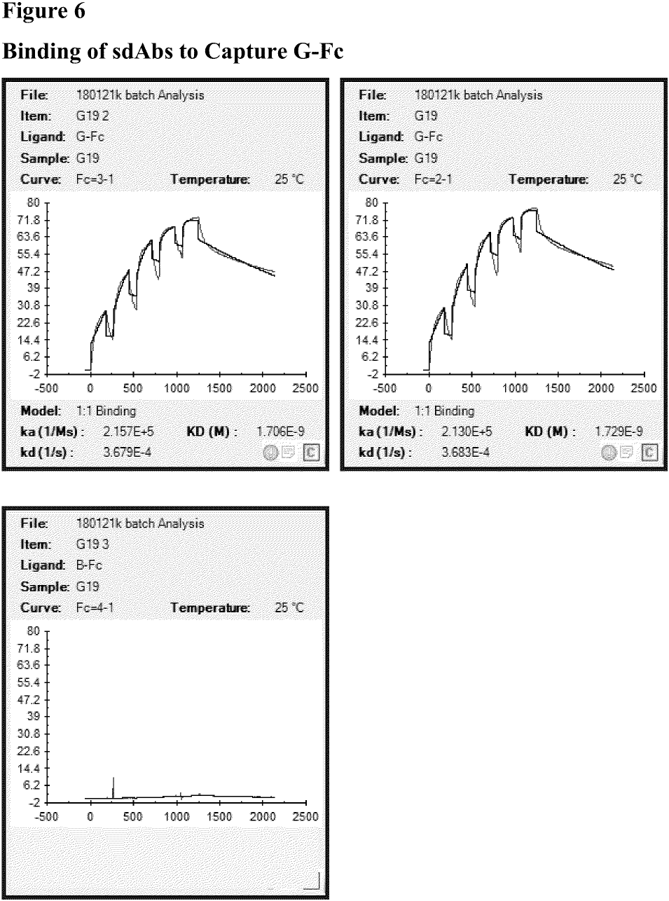

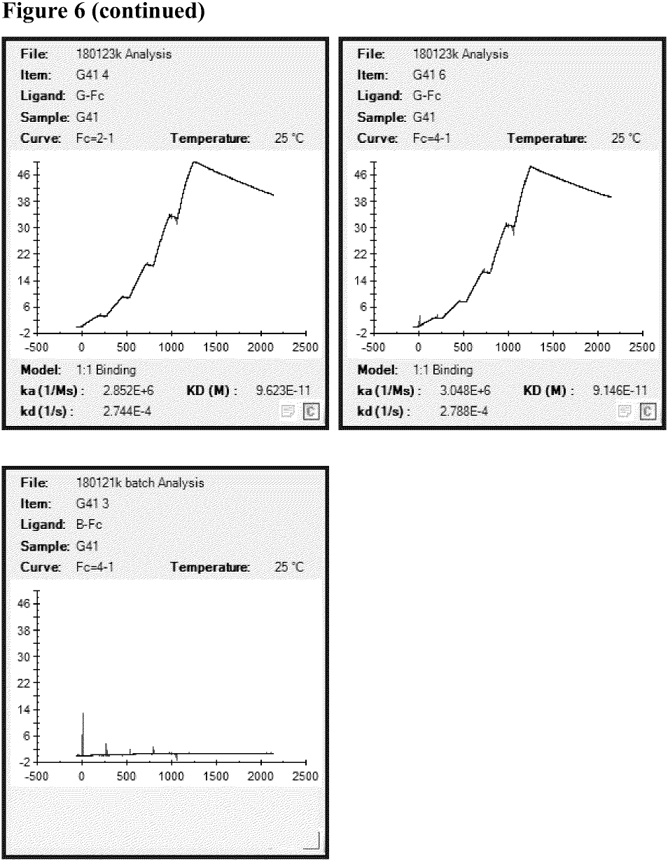

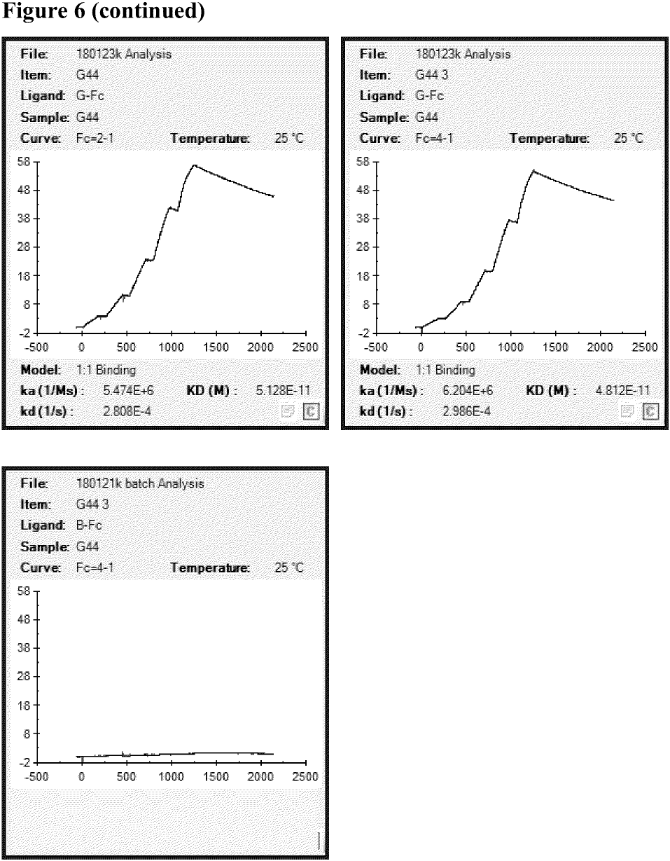

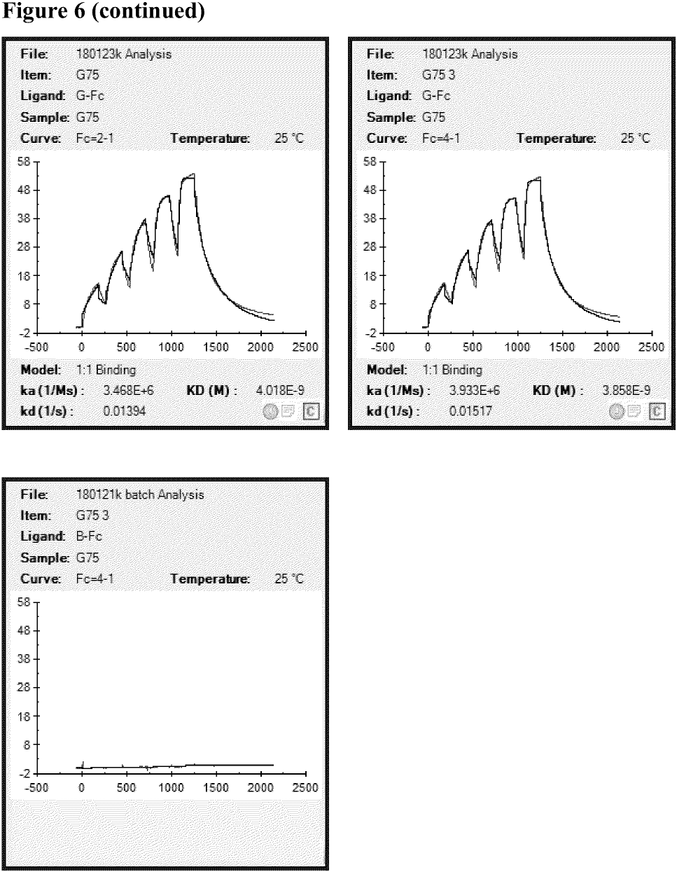

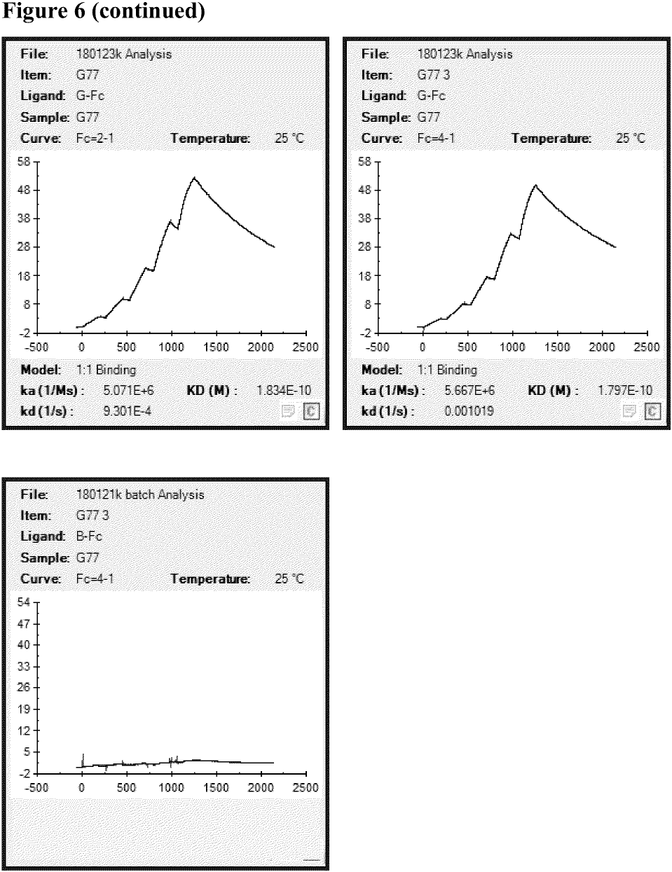

Surfaces were regenerated with a 120 s injection of 10 mM glycine, pH 1.5, at a flow rate of 30 µL/min. Reference flow cell subtracted sensorgrams were fit to a 1:1 binding model to determine kinetics and affinities using the BIAevaluation Software v3.0 (Cytiva). The 5 VHHs bound glycoprotein D, with most demonstrating high affinity binding with K_{DS} ranging 50 pM to 4 nM (Table 6 and FIG. 5). One VHH (G41) bound but could not be fit to a 1:1 interaction model. Certain SRP sensorgram results are shown in FIG. 6.

TABLE 6

Kinetic Analysis Data

| Sample | Capture (RU) | Ligand | Rmax (RU) | Chi2 (RU2) | U-value | ka (1/Ms) | kd (1/s) | KD (nM) | Notes |
|---|---|---|---|---|---|---|---|---|---|
| G19 | 337 | G-Fc | | | | | | | Poor fit to 1:1 model |
| | 309 | G-Fc | | | | | | | Poor fit to 1:1 model |
| G41 | 272 | G-Fc | 63 | 0.0319 | 1 | 2.85E+0 6 | 2.74E-04 | 0.096 | |
| | 272 | G-Fc | 62.5 | 0.0846 | 1 | 3.05E+0 6 | 2.79E-04 | 0.092 | |
| G44 | 269 | G-Fc | 61.6 | 0.032 | 1 | 5.47E+0 6 | 2.81E-04 | 0.051 | |
| | 267 | G-Fc | 59.9 | 0.0859 | 1 | 6.20E+0 6 | 2.99E-04 | 0.048 | |
| G75 | 262 | G-Fc | 57.1 | 2.17 | 5 | 3.47E+0 6 | 1.39E-02 | 4.018 | |
| | 261 | G-Fc | 55.6 | 1.8 | 5 | 3.93E+0 6 | 1.52E-02 | 3.858 | |
| G77 | 266 | G-Fc | 62.1 | 0.0177 | 1 | 5.07E+0 6 | 9.30E-04 | 0.183 | |
| | 264 | G-Fc | 60 | 0.141 | 1 | 5.67E+0 6 | 1.02E-03 | 0.180 | |

As shown in Table 6, G41, G44 and G77 showed extremely strong binding to G-Fc with affinities (K_{D}) ranging from 0.05 - 0.2 nM. G75 showed strong binding to G-Fc with a KD of approximately 4 nM. G19 showed specific binding to G-Fc; however, it could not be fit to a 1:1 binding model. A heavy precipitate was observed when this sample was thawed before performing the SEC purification.

SPR was also used to perform epitope binning, to determine how many overlapping or non-overlapping epitopes were targeted by the 5 glycoprotein D VHHs: under the following conditions:
Capture of targets B-Fc and G-Fc on Fc 2, 3 & 4: flow rate: 10 AL/min, injection time: variable (90 - 135 s),
concentration: B-Fc at 5 Ag/mL and G-Fc at 1 ug/mL
Dual injection (co-injection) of SEC purified sdAbs on Fc 1-4: flow rate: 30
AL/min, injection volume: 120 uL
Concentrations: all sdAbs were injected at 25×KD
Injection 1: sdAb A followed by injection 2: sdAb A + sdAb B
Regeneration: flow rate: 30 uL/min, 60 uL (120 s) 10 mM glycine, pH 1.5

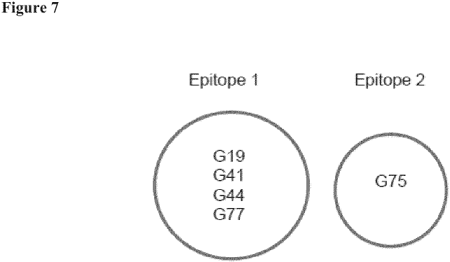

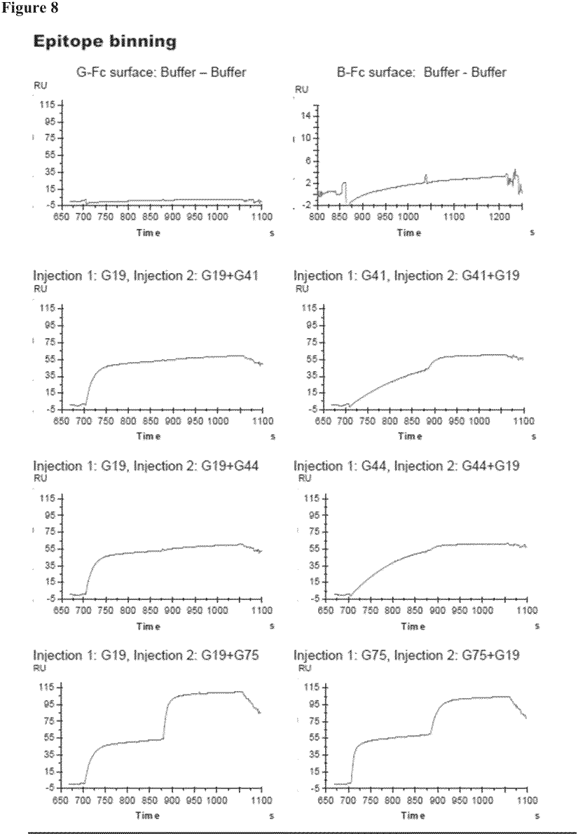

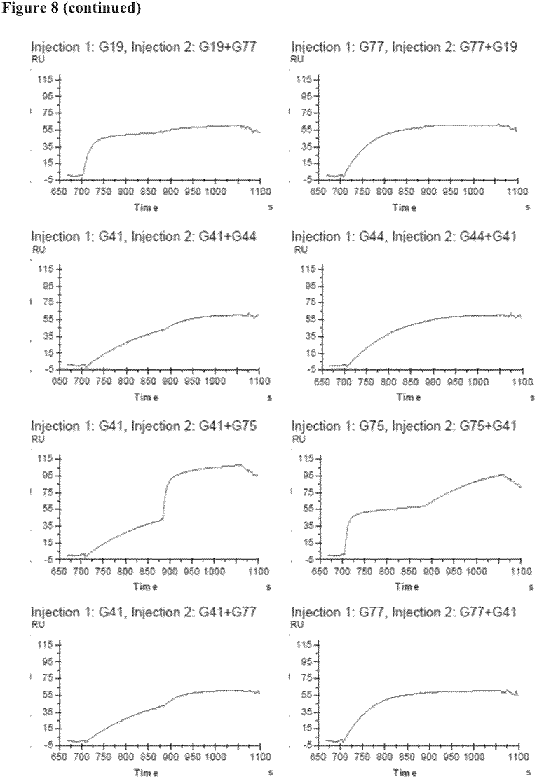

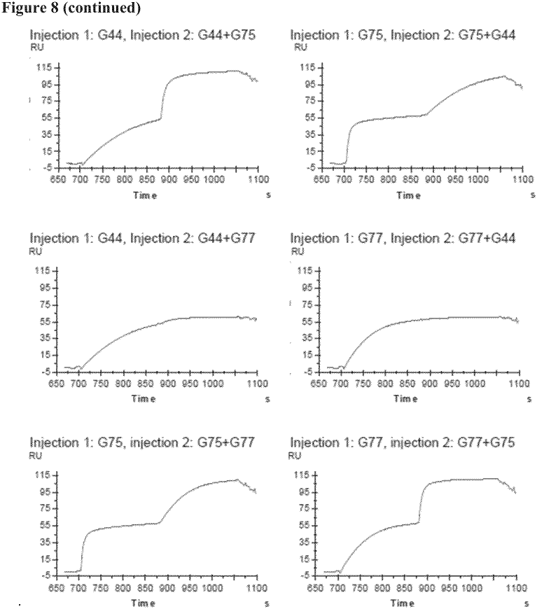

Using captured G-Fc antigen as described above, pairs of VHHs were injected in sequence at surface-saturating concentrations. Injection 1 consisted of VHH1 (at 25× KD concentration) for 180 s, followed by injection 2 which was a mix of VHH1 + VHH2 (both at 25× KD concentration) for 180 s, all at a flow rate of 30 µL/min. The opposite orientation (VHH2, followed by VHH2 + VHH1) was also performed. Surfaces were regenerated using conditions described above. Based on the response of the second injection (or lack thereof), all VHH pairs were mapped into overlapping or non-overlapping epitope bins and this is shown graphically in FIG. 7. Two different epitopes were found on G-Fc: G19, G41, G44 and G77 bound to a different epitope than G75. SPR epitope binning results are shown in FIG. 8.

### Example 11 In Vitro Neutralization Assays

Purified glycoprotein D-specific VHHs were tested for HSV-1 neutralization ability by exposing the HSV-1 virus to different antibodies, followed by testing the infectivity of the HSV-1. Briefly, VG17 virus (HSV-1 wildtype non-purified virus, titer of ~2.0×10E+8) was diluted 100,000× (10 ul in 1 ml raw DMEM, then 10 ul in 10 ml raw DMEM). Three µgs of each antibody was mixed with 50 ul of the diluted virus and the volume was adjusted to 1 ml using raw DMEM medium. The mixture was incubated for 1 hour at room temperature, after which, 250 ul of each dilution was used to infect VERO cells in 12 well plate.

The virus was allowed to infect the cells for 1 hour, followed by removal of the medium, washing with 1 ml acidified PBS then 1 ml PBS. The cells were covered by adding 1 ml of methyl cellulose in DMEM each well. From all tested antibodies, only G75 antibody possessed the ability to neutralize or reduce the infectivity of the HSV-1 virus.

Further exemplary embodiments are illustrated below.

Embodiment 1. An antibody comprising a heavy chain variable region, wherein the heavy chain variable region comprises three complementarity determining regions (CDRs) CDR1, CDR2, and CDR3, wherein the CDR1 is selected from SEQ ID NOs: 17-25, the CDR2 is selected from SEQ ID NOs:30-36, and the CDR3 is selected from SEQ ID NOs:45-63, and wherein the antibody binds a glycoprotein D (gD protein).

Embodiment 2. The antibody of Embodiment 1, wherein the CDR1, CDR2, and CDR3 comprise:
(a) SEQ ID NO: 17, SEQ ID NO:30, and SEQ ID NO:45, respectively;
(b) SEQ ID NO: 17, SEQ ID NO:31, and SEQ ID NO:46, respectively;
(c) SEQ ID NO: 18, SEQ ID NO:32, and SEQ ID NO:47, respectively;
(d) SEQ ID NO: 19, SEQ ID NO:31, and SEQ ID NO:48, respectively;
(e) SEQ ID NO:20, SEQ ID NO:33, and SEQ ID NO:45, respectively;
(f) SEQ ID NO:21, SEQ ID NO:31, and SEQ ID NO:49, respectively;
(g) SEQ ID NO:18, SEQ ID NO:32, and SEQ ID NO:50, respectively;
(h) SEQ ID NO:18, SEQ ID NO:32, and SEQ ID NO:51, respectively;
(i) SEQ ID NO:22, SEQ ID NO:31, and SEQ ID NO:48, respectively;
(j) SEQ ID NO:22, SEQ ID NO:31, and SEQ ID NO:52, respectively;
(k) SEQ ID NO:23, SEQ ID NO:31, and SEQ ID NO:53, respectively;
(l) SEQ ID NO: 18, SEQ ID NO:32, and SEQ ID NO:54, respectively;
(m) SEQ ID NO: 17, SEQ ID NO:32, and SEQ ID NO:55, respectively;
(n) SEQ ID NO: 17, SEQ ID NO:32, and SEQ ID NO:56, respectively;
(o) SEQ ID NO:22, SEQ ID NO:31, and SEQ ID NO:57, respectively;
(p) SEQ ID NO:22, SEQ ID NO:31, and SEQ ID NO:58, respectively;
(q) SEQ ID NO:18, SEQ ID NO:32, and SEQ ID NO:59, respectively;
(r) SEQ ID NO: 17, SEQ ID NO:34, and SEQ ID NO:60, respectively;
(s) SEQ ID NO:24, SEQ ID NO:35, and SEQ ID NO:61, respectively;
(t) SEQ ID NO:22, SEQ ID NO:31, and SEQ ID NO:62, respectively; or
(u) SEQ ID NO:25, SEQ ID NO:36, and SEQ ID NO:63, respectively.

Embodiment 3. The antibody of any one of Embodiments 1-2, wherein the antibody comprises a framework region 1 (FR1) selected from SEQ ID NOs: 1-16, a framework region 2 (FR2) selected from SEQ ID NOs:26-29, a framework region 3 (FR3) selected from SEQ ID NOs:37-44, and a framework region 4 (FR4) selected from SEQ ID NOs:64-66.

Embodiment 4. The antibody of any one of Embodiments 1-3, wherein the FR1, FR2, FR3 and FR4 comprise:
(a) SEQ ID NO:1, SEQ ID NO:26, SEQ ID NO:37, and SEQ ID NO:64, respectively;
(b) SEQ ID NO:2, SEQ ID NO:26, SEQ ID NO:38, and SEQ ID NO:64, respectively;
(c) SEQ ID NO:3, SEQ ID NO:26, SEQ ID NO:39, and SEQ ID NO:64, respectively;
(d) SEQ ID NO:4, SEQ ID NO:26, SEQ ID NO:40, and SEQ ID NO:65, respectively;
(e) SEQ ID NO:5, SEQ ID NO:27, SEQ ID NO:37, and SEQ ID NO:64, respectively;
(f) SEQ ID NO:6, SEQ ID NO:26, SEQ ID NO:41, and SEQ ID NO:64, respectively;
(g) SEQ ID NO:7, SEQ ID NO:26, SEQ ID NO:39, and SEQ ID NO:64, respectively;
(h) SEQ ID NO:8, SEQ ID NO:26, SEQ ID NO:39, and SEQ ID NO:64, respectively;
(i) SEQ ID NO:9, SEQ ID NO:26, SEQ ID NO:40, and SEQ ID NO:65, respectively;
(j) SEQ ID NO:2, SEQ ID NO:26, SEQ ID NO:40, and SEQ ID NO:64, respectively;
(k) SEQ ID NO:10, SEQ ID NO:26, SEQ ID NO:41, and SEQ ID NO:64, respectively;
(l) SEQ ID NO:8, SEQ ID NO:26, SEQ ID NO:39, and SEQ ID NO:64, respectively;
(m) SEQ ID NO:11, SEQ ID NO:26, SEQ ID NO:38, and SEQ ID NO:64, respectively;
(n) SEQ ID NO:12, SEQ ID NO:26, SEQ ID NO:38, and SEQ ID NO:64, respectively;
(o) SEQ ID NO:13, SEQ ID NO:26, SEQ ID NO:41, and SEQ ID NO:64, respectively;
(p) SEQ ID NO:2, SEQ ID NO:26, SEQ ID NO:42, and SEQ ID NO:64, respectively;
(q) SEQ ID NO:8, SEQ ID NO:26, SEQ ID NO:39, and SEQ ID NO:64, respectively;
(r) SEQ ID NO:12, SEQ ID NO:26, SEQ ID NO:38, and SEQ ID NO:64, respectively;
(s) SEQ ID NO:14, SEQ ID NO:28, SEQ ID NO:43, and SEQ ID NO:66, respectively;
(t) SEQ ID NO:15, SEQ ID NO:26, SEQ ID NO:41, and SEQ ID NO:64, respectively; or
(u) SEQ ID NO:16, SEQ ID NO:29, SEQ ID NO:44, and SEQ ID NO:64, respectively.

Embodiment 5. The antibody of any one of Embodiments 1-4, wherein the antibody comprises an amino acid sequence with at least 80%, 85%, 90%, 95%, 96%, 97%, 98%, 99%, or 100% identity to any one of SEQ ID NOs:67-87.

Embodiment 6. The antibody of any one of Embodiments 1-5, comprising a heavy chain variable region, wherein the heavy chain variable region comprises an amino acid sequence as set forth in any one of SEQ ID NOs:67-87.

Embodiment 7. The antibody of any one of Embodiments 1-6, wherein the gD protein is derived from a virus.

Embodiment 8. The antibody of Embodiment 7, wherein the virus is a Herpes Simplex Virus (HSV).

Embodiment 9. The antibody of Embodiment 8, wherein the HSV is an HSV-1 or an HSV-2.

Embodiment 10. The antibody of any one of Embodiments 1-9, wherein the gD protein is a recombinant gD protein.

Embodiment 11. The antibody of any one of Embodiments 1-10, wherein the antibody binds HSV-1 and/or HSV-2.

Embodiment 12. The antibody of any one of Embodiments 1-11, wherein the antibody binds the gD protein with a K_{D} ranging from about 1 pM to about 1 µM.

Embodiment 13. The antibody of any one of Embodiments 1-12, wherein the K_{D} ranges from about 50 pM to about 4 nM.

Embodiment 14. The antibody of any one of Embodiments 1-13, wherein the K_{D} ranges from 0.05 nM to 0.2 nM.

Embodiment 15. The antibody of any one of Embodiments 1-14, wherein the antibody binds to the gD protein with a K_{D} of about 4 nM or lower.

Embodiment 16. The antibody of any one of Embodiments 1-15, wherein the antibody is a single heavy domain antibody (sdAb) or a multi-specific antibody.

Embodiment 17. The antibody of any one of Embodiments 1-16, wherein the antibody is a neutralizing antibody.

Embodiment 18. The antibody of any one of Embodiments 1-17, wherein the antibody is capable of neutralizing HSV-1 and/or HSV-2.

Embodiment 19. The antibody of any one of Embodiments 17-18, wherein the antibody comprises an amino acid sequence with at least 90%, 95%, 96%, 97%, 98%, 99%, or 100% identity to SEQ ID NO:85.

Embodiment 20. The antibody of any one of Embodiments 1-16, wherein the antibody is a non-neutralizing antibody.

Embodiment 21. The antibody of any one of Embodiments 1-16, wherein the antibody is a multi-specific antibody comprising an amino acid sequence set forth in SEQ ID NO:85 and one or more amino acid sequences selected from SEQ ID NOs:67-84 and 86-87.

Embodiment 22. A composition comprising the antibody of any one of Embodiments 1-21.

Embodiment 23. A pharmaceutical composition comprising the antibody of any one of Embodiments 1-21 and a pharmaceutically acceptable carrier.

Embodiment 24. A polynucleotide encoding the antibody of any one of Embodiments 1-21.

Embodiment 25. A vector comprising the polynucleotide of Embodiment 24.

Embodiment 26. A cell capable of expressing the antibody of any one of Embodiments 1-25.

Embodiment 27. A cell comprising the polynucleotide of Embodiment 24 or the vector of Embodiment 25.

Embodiment 28. A method of producing an antibody, comprising culturing the cell of Embodiment 26 or 27 and recovering the antibody from the cell.

Embodiment 29. A method for treating infection of HSV-1 and/or HSV-2 in a subject, comprising administering to the subject an effective amount of the antibody of any one of Embodiments 1-21 or the composition of Embodiment 22 or 23.

Embodiment 30. A method for detecting HSV-1 and/or HSV-2 in a sample, comprising contacting the sample with the antibody of any one of Embodiments 1-21 or the composition of Embodiment 22 or 23, and detecting the presence of the antibody.

While the disclosure has been particularly shown and described with reference to specific embodiments, it should be understood by those having skill in the art that various changes in form and detail may be made therein without departing from the spirit and scope of the present disclosure as disclosed herein.

### REFERENCE LIST

1. Henry KA, van Faassen H, Harcus D, et al. Llama peripheral B-cell populations producing conventional and heavy chain-only IgG subtypes are phenotypically indistinguishable but immunogenetically distinct. Immunogenetics. 2019;71(4):307-320.doi:10.1007/s00251-018-01102-9
2. Baral et al Curr Protoc Immunol Single-domain antibodies and their utility - PubMed (nih.gov)
3. Hussack G, Arbabi-Ghahroudi M, van Faassen H, et al. Neutralization of Clostridium difficile toxin A with single-domain antibodies targeting the cell receptor binding domain. J Biol Chem. 2011;286(11):8961-8976. doi: 10.1074/jbc.M110.198754
4. Safety and Efficacy of Oncolytic HSV-1 G207 Inoculated into the Cerebellum of Mice (nih.gov). Cancer Gene Ther. Author manuscript; available in PMC 2020 Oct 1. Published in final edited form as: Cancer Gene Ther. 2020 Apr; 27(3-4): 246-255. Published online 2019 Mar 28. doi: 10.1038/s41417-019-0091-0. https://pubmed.ncbi.nlm.nih.gov/15903252/
5. Agrawal V, Slivac I, Perret S, et al. Stable expression of chimeric heavy chain antibodies in CHO cells. Methods Mol Biol. 2012;911:287-303. doi: 10.1007/978-1-61779-968-6_18
6. Batzer, M.A., Arcot, S. S., Phinney, J.W., Alegria-Hartman, M., Kass, D.H., & Milligan, S.M., et al. (1996). Genetic variation of recent alu insertions in human populations. Journal of Molecular Evolution, 42(1), 22-29.
7. Ohtsuka, E., Matsuki, S., Ikehara, M., Takahashi, Y., & Matsubara, K. (1985). An alternative approach to deoxyoligonucleotides as hybridation probes by insertion of deoxyinosine at ambiguous codon positions. Journal of Biological Chemistry, 260(5), 2605-2608.
8. Cassol, S.A., Lapointe, N., Salas, T., Hankins, C., & Charest, J. (1992). Diagnosis of vertical hiv-1 transmission using the polymerase chain reaction and dried blood spot specimens. J Acquir Immune Defic Syndr, 5(2), 113-119.
9. Rossolini, G.M., Cresti, S., Ingianni, A., Cattani, P., & Satta, G. (1994). Use of deoxyinosine-containing primers vs degenerate primers for polymerase chain reaction based on ambiguous sequence information. Molecular & Cellular Probes, 8(2), 91.
10. “Sequences of Proteins of Immunological Interest,” 5th Ed. Public Health Service, National Institutes of Health, Bethesda, MD (“Kabat” numbering scheme),
11. Al-Lazikani et al., (1997) JMB 273,927-948 (“Chothia” numbering scheme);
12. MacCallum et al., J. Mol. Biol. 262:732-745 (1996), “Antibody-antigen interactions: Contact analysis and binding site topography,” J. Mol. Biol. 262, 732-745.” (“Contact” numbering scheme);
13. Lefranc MP et al., “IMGT unique numbering for immunoglobulin and T cell receptor variable domains and Ig superfamily V-like domains,” Dev Comp Immunol, 2003 Jan;27(1):55-77 (“IMGT” numbering scheme);
14. Honegger A and Plückthun A, “Yet another numbering scheme for immunoglobulin variable domains: an automatic modeling and analysis tool,” J Mol Biol, 2001 Jun 8;309(3):657-70, (“Aho” numbering scheme);
15. Martin et al., “Modeling antibody hypervariable loops: a combined algorithm,” PNAS, 1989, 86(23):9268-9272, (“AbM” numbering scheme).
16. Pope M.E., Soste M.V., Eyford B.A., Anderson N.L., Pearson T.W., (2009) J. Immunol. Methods. 341(1-2):86-96
17. Polpitiya Arachchige, S., Henke, W., Kalamvoki, M. et al. Analysis of herpes simplex type 1 gB, gD, and gH/gL on production of infectious HIV-1: HSV-1 gD restricts HIV-1 by exclusion of HIV-1 Env from maturing viral particles. Retrovirology 16, 9 (2019).
18. Karasneh G. A., Shukla D. (2011). Herpes simplex virus infects most cell types in vitro: Clues to its success. Virol. J. 8:481. doi: 10.1186/1743-422X-8-481
19. Carson J, Haddad D, Bressman M, Fong Y. ONCOLYTIC HERPES SIMPLEX VIRUS 1 (HSV-1) VECTORS: INCREASING TREATMENT EFFICACY AND RANGE THROUGH STRATEGIC VIRUS DESIGN. Drugs Future. 2010;35(3):183-195. doi:10.1358/dof.2010.35.3.1470166
20. Surface Plasmon Resonance, Nico J. MolMarcel J. E. Fischer, DOI:10.1007/978-1-60761-670-2 ISBN: 978-1-60761-669-6.
21. Kaslow, R. A.; Stanberry, L.R.; Le Duc, J. W., eds. (2014). Viral Infections of Humans: Epidemiology and Control (5th ed.). Springer. p. 56. ISBN 9781489974488
22. Construction of a Fully Retargeted Herpes Simplex Virus 1 Recombinant Capable of Entering Cells Solely via Human Epidermal Growth Factor Receptor 2, Laura Menotti et al., Jounal of Virology, 12.22.2020, DOI: https://doi.org/10.1128/JVI.01133-08.
23. Hermann K., Ollert M., Ring J. (2005) Antibody detection. In: Nijkamp F.P., Parnham M.J. (eds) Principles of Immunopharmacology. Birkhäuser Basel. https://doi.org/10.1007/3-7643-7408-X_11
24. Baldi L, Muller N, Picasso S, et al. Transient gene expression in suspension HEK-293 cells: application to large-scale protein production. Biotechnol Prog. 2005;21(1):148-153. doi:10.1021/bp049830x
25. Attenuated multi-mutated herpes simplex virus-1 for the treatment of malignant gliomas. Mineta T, Rabkin SD, Yazaki T, Hunter WD, Martuza RL Nat Med. 1995 Sep; 1(9):938-43.
26. Serial passage through human glioma xenografts selects for a Deltagamma134.5 herpes simplex virus type 1 mutant that exhibits decreased neurotoxicity and prolongs survival of mice with experimental brain tumors. Shah AC, Price KH, Parker JN, Samuel SL, Meleth S, Cassady KA, Gillespie GY, Whitley RJ, Markert JM, J Virol. 2006 Aug; 80(15):7308-15.
27. Treatment of intracranial gliomas in immunocompetent mice using herpes simplex viruses that express murine interleukins. Andreansky S, He B, van Cott J, McGhee J, Markert JM, Gillespie GY, Roizman B, Whitley RJ, Gene Ther. 1998 Jan; 5(1): 121-30.

What is claimed is:

1. An antibody comprising a heavy chain variable region, wherein the heavy chain variable region comprises three complementarity determining regions (CDRs) CDR1, CDR2, and CDR3, wherein the CDR1 is selected from SEQ ID NOs: 17-25, the CDR2 is selected from SEQ ID NOs:30-36, and the CDR3 is selected from SEQ ID NOs:45-63, and wherein the antibody binds a glycoprotein D (gD protein).

2. The antibody of claim 1, wherein the CDR1, CDR2, and CDR3 comprise:
(a) SEQ ID NO: 17, SEQ ID NO:30, and SEQ ID NO:45, respectively;
(b) SEQ ID NO: 17, SEQ ID NO:31, and SEQ ID NO:46, respectively;
(c) SEQ ID NO:18, SEQ ID NO:32, and SEQ ID NO:47, respectively;
(d) SEQ ID NO: 19, SEQ ID NO:31, and SEQ ID NO:48, respectively;
(e) SEQ ID NO:20, SEQ ID NO:33, and SEQ ID NO:45, respectively;
(f) SEQ ID NO:21, SEQ ID NO:31, and SEQ ID NO:49, respectively;
(g) SEQ ID NO: 18, SEQ ID NO:32, and SEQ ID NO:50, respectively;
(h) SEQ ID NO:18, SEQ ID NO:32, and SEQ ID NO:51, respectively;
(i) SEQ ID NO:22, SEQ ID NO:31, and SEQ ID NO:48, respectively;
(j) SEQ ID NO:22, SEQ ID NO:31, and SEQ ID NO:52, respectively;
(k) SEQ ID NO:23, SEQ ID NO:31, and SEQ ID NO:53, respectively;
(l) SEQ ID NO: 18, SEQ ID NO:32, and SEQ ID NO:54, respectively;
(m) SEQ ID NO: 17, SEQ ID NO:32, and SEQ ID NO:55, respectively;
(n) SEQ ID NO: 17, SEQ ID NO:32, and SEQ ID NO:56, respectively;
(o) SEQ ID NO:22, SEQ ID NO:31, and SEQ ID NO:57, respectively;
(p) SEQ ID NO:22, SEQ ID NO:31, and SEQ ID NO:58, respectively;
(q) SEQ ID NO: 18, SEQ ID NO:32, and SEQ ID NO:59, respectively;
(r) SEQ ID NO: 17, SEQ ID NO:34, and SEQ ID NO:60, respectively;
(s) SEQ ID NO: 24, SEQ ID NO:35, and SEQ ID NO:61, respectively;
(t) SEQ ID NO:22, SEQ ID NO:31, and SEQ ID NO:62, respectively; or
(u) SEQ ID NO:25, SEQ ID NO:36, and SEQ ID NO:63, respectively.

3. The antibody of claim 2, wherein the antibody comprises a framework region 1 (FR1) selected from SEQ ID NOs: 1-16, a framework region 2 (FR2) selected from SEQ ID NOs:26-29, a framework region 3 (FR3) selected from SEQ ID NOs:37-44, and a framework region 4 (FR4) selected from SEQ ID NOs:64-66.

4. The antibody of claim 3, wherein the FR1, FR2, FR3 and FR4 comprise:
(a) SEQ ID NO:1, SEQ ID NO:26, SEQ ID NO:37, and SEQ ID NO:64, respectively;
(b) SEQ ID NO:2, SEQ ID NO:26, SEQ ID NO:38, and SEQ ID NO:64, respectively;
(c) SEQ ID NO:3, SEQ ID NO:26, SEQ ID NO:39, and SEQ ID NO:64, respectively;
(d) SEQ ID NO:4, SEQ ID NO:26, SEQ ID NO:40, and SEQ ID NO:65, respectively;
(e) SEQ ID NO:5, SEQ ID NO:27, SEQ ID NO:37, and SEQ ID NO:64, respectively;
(f) SEQ ID NO:6, SEQ ID NO:26, SEQ ID NO:41, and SEQ ID NO:64, respectively;
(g) SEQ ID NO:7, SEQ ID NO:26, SEQ ID NO:39, and SEQ ID NO:64, respectively;
(h) SEQ ID NO:8, SEQ ID NO:26, SEQ ID NO:39, and SEQ ID NO:64, respectively;
(i) SEQ ID NO:9, SEQ ID NO:26, SEQ ID NO:40, and SEQ ID NO:65, respectively;
(j) SEQ ID NO:2, SEQ ID NO:26, SEQ ID NO:40, and SEQ ID NO:64, respectively;
(k) SEQ ID NO:10, SEQ ID NO:26, SEQ ID NO:41, and SEQ ID NO:64, respectively;
(l) SEQ ID NO:8, SEQ ID NO:26, SEQ ID NO:39, and SEQ ID NO:64, respectively;
(m) SEQ ID NO:11, SEQ ID NO:26, SEQ ID NO:38, and SEQ ID NO:64, respectively;
(n) SEQ ID NO:12, SEQ ID NO:26, SEQ ID NO:38, and SEQ ID NO:64, respectively;
(o) SEQ ID NO:13, SEQ ID NO:26, SEQ ID NO:41, and SEQ ID NO:64, respectively;
(p) SEQ ID NO:2, SEQ ID NO:26, SEQ ID NO:42, and SEQ ID NO:64, respectively;
(q) SEQ ID NO:8, SEQ ID NO:26, SEQ ID NO:39, and SEQ ID NO:64, respectively;
(r) SEQ ID NO:12, SEQ ID NO:26, SEQ ID NO:38, and SEQ ID NO:64, respectively;
(s) SEQ ID NO:14, SEQ ID NO:28, SEQ ID NO:43, and SEQ ID NO:66, respectively;
(t) SEQ ID NO:15, SEQ ID NO:26, SEQ ID NO:41, and SEQ ID NO:64, respectively; or
(u) SEQ ID NO:16, SEQ ID NO:29, SEQ ID NO:44, and SEQ ID NO:64, respectively.

5. The antibody of claim 1, wherein the antibody comprises an amino acid sequence with at least 80%, 85%, 90%, 95%, 96%, 97%, 98%, 99%, or 100% identity to any one of SEQ ID NOs:67-87.

6. The antibody of claim 1, comprising a heavy chain variable region, wherein the heavy chain variable region comprises an amino acid sequence as set forth in any one of SEQ ID NOs:67-87.

7. The antibody of claim 1, wherein the gD protein is derived from a virus.

8. The antibody of claim 7, wherein the virus is a Herpes Simplex Virus (HSV).

9. The antibody of claim 8, wherein the HSV is an HSV-1 or an HSV-2.

10. The antibody of claim 1, wherein the gD protein is a recombinant gD protein.

11. The antibody of claim 1, wherein the antibody binds HSV-1 and/or HSV-2.

12. The antibody of claim 1, wherein the antibody binds the gD protein with a K_{D} ranging from about 1 pM to about 1 µM.

13. The antibody of claim 12, wherein the K_{D} ranges from about 50 pM to about 4 nM.

14. The antibody of claim 13, wherein the K_{D} ranges from 0.05 nM to 0.2 nM.

15. The antibody of claim 1, wherein the antibody binds to the gD protein with a K_{D} of about 4 nM or lower.

16. The antibody of claim 1, wherein the antibody is a single heavy domain antibody (sdAb) or a multi-specific antibody.

17. The antibody of claim 1, wherein the antibody is a neutralizing antibody.

18. The antibody of claim 17, wherein the antibody is capable of neutralizing HSV-1 and/or HSV-2.

19. The antibody of claim 18, wherein the antibody comprises an amino acid sequence with at least 90%, 95%, 96%, 97%, 98%, 99%, or 100% identity to SEQ ID NO:85.

20. The antibody of claim 1, wherein the antibody is a non-neutralizing antibody.

21. The antibody of claim 1, wherein the antibody is a multi-specific antibody comprising an amino acid sequence set forth in SEQ ID NO:85 and one or more amino acid sequences selected from SEQ ID NOs:67-84 and 86-87.

22. A composition comprising the antibody of claim 1.

23. A pharmaceutical composition comprising the antibody of claim 1 and a pharmaceutically acceptable carrier.

24. A polynucleotide encoding the antibody of claim 1.

25. A vector comprising the polynucleotide of claim 24.

26. A cell capable of expressing the antibody of claim 1.

27. A cell comprising the polynucleotide of claim 24 or a cell comprising a vector comprising the polynucleotide of claim 24.

28. A method of producing an antibody, comprising culturing the cell of claim 27 and recovering the antibody from the cell.

29. A method for treating infection of HSV-1 and/or HSV-2 in a subject, comprising administering to the subject an effective amount of the antibody of claim 1.

30. A method for treating infection of HSV-1 and/or HSV-2 in a subject, comprising administering to the subject an effective amount of the pharmaceutical composition of claim 23.

31. A method for detecting HSV-1 and/or HSV-2 in a sample, comprising contacting the sample with the antibody of claim 1, and detecting the presence of the antibody.

32. A method for detecting HSV-1 and/or HSV-2 in a sample, comprising contacting the sample with the pharmaceutical composition of claim 23, and detecting the presence of the antibody.
